(19) United States
(12) Patent Application Publication
(10) Pub. No.: US 2025/0101406 A1
(43) Pub. Date: Mar. 27, 2025

(54) 3D MODELS FOR PREDICTING TREATMENT RESPONSES TO ALTERNATING ELECTRIC FIELDS

(71) Applicant: NOVOCURE GMBH, Root D4 (CH)

(72) Inventors: CARSTEN HAGEMANN, Unterpleichfeld (DE); VERA NICKL, Würzburg (DE); MARIO LOHR, Würzburg (DE); ELLAINE SALVADOR, Würzburg (DE); ALMUTH F. KESSLER, Würzburg (DE)

(21) Appl. No.: 18/472,931

(22) Filed: Sep. 22, 2023

Related U.S. Application Data
(60) Provisional application No. 63/409,525, filed on Sep. 23, 2022.

(51) Int. Cl. C12N 13/00; A61B 5/00; G01N 33/50

(52) U.S. Cl. CPC C12N 13/00; A61B 5/4848; G01N 33/5005

(57) ABSTRACT

Disclosed are methods of determining the efficacy of an alternating electric field comprising applying an alternating electric field to one or more microtumors for a period of time, the alternating electric field having a frequency and field strength, wherein the microtumor comprises primary cancer cells; and determining the efficacy of the alternating electric field. Disclosed are methods of testing the efficacy of an alternating electric field comprising applying alternating electric fields to one or more organoids for a period of time, the alternating electric fields having a frequency and field strength, wherein the organoids are cultured on organotypic hippocampal slice cultures; and determining the efficacy of alternating electric fields. Disclosed are methods of testing the efficacy of alternating electric fields on a subject comprising culturing or incubating one or more tumor slices from the subject, applying alternating electric fields to the one or more tumor slices for a period of time, the alternating electric fields having a frequency and field strength, and determining the efficacy of alternating electric fields.

### CROSS-REFERENCE TO RELATED APPLICATIONS

This application claims the benefit of U.S. Provisional Patent Application No. 63/409,525, filed Sep. 23, 2022, which is incorporated by reference herein in its entirety.

### BACKGROUND

In glioblastoma (GBM), tumor recurrence is inevitable and the prognosis of patients is poor, despite multidisciplinary treatment approaches involving surgical resection, radiotherapy and chemotherapy. Recently, Tumor Treating Fields (TTFields) have been added to the therapeutic set-up. These alternating electric fields are applied to glioblastoma at 200 kHz frequency via arrays placed on the shaved scalp of patients. Patients show varying response to this therapy. Molecular effects of TTFields have been investigated largely in cell cultures and animal models, but not in patients' tissue samples. Acquisition of matched treatment-naïve and recurrent patient tissues is a challenge. Therefore, discussed herein are three reliable patient-derived 3-dimensional ex vivo models (primary cells grown as microtumors on murine organotypic hippocampal slices, organoids and tumor slice cultures) which can facilitate prediction of patients' treatment response and provide important insights into clinically relevant cellular and molecular alterations under TTFields.

### BRIEF SUMMARY

Disclosed are methods of determining the efficacy of an alternating electric field using one or more of three different ex vivo 3D models: microtumors, organoids, tumor slices.

Disclosed are methods of determining the efficacy of an alternating electric field comprising applying an alternating electric field to one or more microtumors for a period of time, the alternating electric field having a frequency and field strength, wherein the microtumor comprises primary cancer cells; and determining the efficacy of the alternating electric field. In some aspects, the method can be a method of determining the efficacy of an alternating electric field for the treatment of cancer, comprising exposing a microtumor to an alternating electric field; and determining the effect of the alternating electric field on the microtumor.

Disclosed are methods of testing the efficacy of an alternating electric field comprising applying alternating electric fields to one or more organoids for a period of time, the alternating electric fields having a frequency and field strength, wherein the organoids are cultured on organotypic hippocampal slice cultures; and determining the efficacy of alternating electric fields.

Disclosed are methods of testing the efficacy of alternating electric fields on a subject comprising culturing or incubating one or more tumor slices from the subject, applying alternating electric fields to the one or more tumor slices for a period of time, the alternating electric fields having a frequency and field strength, and determining the efficacy of alternating electric fields.

Disclosed are methods of determining or selecting a value of a parameter of an alternating electric field to be administered for treating a disease or condition, the method comprising applying an alternating electric field to at least two microtumors produced from cells obtained from a subject, wherein the alternating electric field applied to each of the at least two microtumors has a different frequency from the other, and determining which frequency is most effective at reducing viability of cancer cells in the microtumors.

Disclosed are methods of determining or selecting a value of a parameter of an alternating electric field to be administered for treating a disease or condition, the method comprising applying an alternating electric field to at least two organoids produced from tissue obtained from a subject, wherein the alternating electric field applied to each of the at least two organoids has a different frequency from the other, and determining which frequency is most effective at reducing viability of cancer cells in the organoids.

Disclosed are methods of determining or selecting a value of a parameter of an alternating electric field to be administered for treating a disease or condition, the method comprising applying an alternating electric field to at least two of a plurality of tumor slices obtained from the tumor of a subject, wherein the alternating electric field applied to each of the at least two tumor slices has a different frequency from the other, and determining which frequency is most effective at reducing viability of cancer cells in the tumor slices.

Disclosed are methods of treating a subject having cancer comprising determining the efficacy of alternating electric fields in an ex vivo model derived from the subject, and applying alternating electric fields to the subject when the alternating electric fields are determined to be effective in the ex vivo model.

Disclosed are methods of identifying biomarkers that respond to application of an alternating electric field comprising performing a morphological or molecular analysis of a microtumor formed from primary cancer cells obtained from a subject, an organoid derived from tissue from a subject, or a tumor slice from a subject; applying an alternating electric field to the microtumor, organoid or tumor slice for a period of time, the alternating electric field having a frequency and field strength; performing a morphological or molecular analysis of the microtumor, organoid or tumor slice after the step of applying an alternating electric field to the microtumor, organoid or tumor slice; and comparing the morphological or molecular analysis of performing a morphological or molecular analysis of the microtumor, organoid or tumor slice prior to applying alternating electric fields with the morphological or molecular analysis performed after applying alternating electric fields, wherein a difference in the morphological or molecular analysis of before applying alternating electric fields with the morphological or molecular analysis of after applying alternating electric fields identifies a biomarker that responds to application of an alternating electric field.

Additional advantages of the disclosed methods and compositions will be set forth in part in the description which follows, and in part will be understood from the description, or may be learned by practice of the disclosed methods and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed methods and compositions.

FIGS. 1A-1G show an example preparation of GBM organoids. A) Intraoperatively obtained tumor tissue was stored on ice in Hibernate A medium. B-D) Under a stereo microscope, the tumor was minced into pieces of 0.5 mm, which were cultured. E) The edges rounded up and organoids formed after two weeks. F) Perfectly formed organoid. Organoids can be cultured for several weeks, propagated by mincing into 100 μm large pieces and re-grown or stored frozen in liquid nitrogen. G) H/E staining of a cross-section through an organoid after 4 weeks of culture.

FIGS. 2A-2G show an example preparation of organotypic hippocampal brain slice cultures (OHSC) and patient derived tumor slices. A) Vibratome and instruments required for the generation of tissue slices. B) Histoacryl glue was placed onto the vibratomes' sample tube. C) The brain and tumor, respectively, were glued on edge into the tube and D) filled with molten agarose. E) The agarose was hardened with a cooling block pre-cooled at −80° C. F) The vibratome was used to generate slices of 350 μm thickness. G) The tumor slices were encased in agarose.

FIGS. 3A-3I show an example sample preparation for TTFields treatment. A) Holder for the inserts with the semipermeable membranes (0.4 μm pore size). B) The insert was placed into the holder. C) Glass coverslip (12 mm diameter) to protect the slices from condensation water. D) The holder with the insert was placed into a ceramic dish with high walls. E) The ceramic dish, but not the insert, was filled with 2.5 ml of brain slice medium. F) An OHSC or tumor slice was transferred using a wide glass pipette and the excess medium was aspirated. G) The slice should be centrally placed on the membrane of the insert. H) Coverslip, parafilm and lid covered the ceramic dish which was I) connected to the base plate for TTFields application.

FIGS. 4A-4C show variable responses of patient derived GBM primary cells to TTFields treatment. A) Cells were grown as monolayer, treated with TTFields at 200 kHz for 72 h and counted (n=3-18). B) Cells were seeded onto organotypic hippocampal slice cultures (OHSC) and grew to microtumors within 3 days (0 h) as visualized by fluorescence microscopy (green). Control cells remained untreated for 72 h (Control 72 h); whereas TTFields treated cells were subjected to TTFields at 200 kHz for 72 h (TTFields 72 h). Scale bar=250 μm. Shown are representative images of U87MG (n=24), GBM-1 (n=7), GBM-2 (n=7), and GBM-3 (n=13). C) Quantification of the microtumor growth on OHSC. A minimum of 3 and up to 83 microtumors were measured. For clinical details of the patient derived primary cells refer to Table 1.

FIGS. 5A and 5B show patient derived GBM-organoid size is reduced by TTFields treatment. A) Comparison of different patient derived GBM organoids, grown on organotypic hippocampal slice cultures (OHSC), before (TTFields 0 h) and after treatment with TTFields at 200 kHz for 72 h (TTFields 72 h). Organoids were visualized by fluorescence microscopy (green). Shown are representative images of n=6. Scale bar=250 μm. B) Quantification of the organoid-growth on OHSC. A minimum of 3 and up to 6 organoids were measured. n.s. =not significant. For clinical details of the patient derived organoids refer to Table 1.

FIGS. 6A-6D show decreased proliferation of TTFields treated human organotypic GBM tumor slice cultures. Human GBM were sliced, cultured, treated with TTFields at 200 kHz for the indicated time periods and A) hematoxylin and cosin stained. B) Immunofluorescence staining of adjacent slices of the same tumors for Ki67 (red), GFAP (green) and with DAPI (blue). Shown are representative images of n=3. Scale bar=100 μm. n.d.=not determined. C) Quantification of Ki67 positive cells in slice cultures of GBM-7 and D) GBM-8 after treatment with TTFields at 200 kHz. For clinical details of the patient derived tumor slices refer to Table 1.

### DETAILED DESCRIPTION

The disclosed methods and compositions may be understood more readily by reference to the following detailed description of particular embodiments and the Example included therein and to the Figures and their previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a microtumor” includes a plurality of such microtumors, reference to “the microtumor” is a reference to one or more microtumors and equivalents thereof known to those skilled in the art, and so forth.

As used herein, a “target site” is a specific site or location within or present on a subject or patient. For example, a “target site” can refer to, but is not limited to a cell (e.g., a cancer cell), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the brain, ovary, lung, pancreas, liver, stomach. In some aspects, a cell or population of cells that can be a target site or a target cell include, but are not limited to, a cancer cell (e.g., an ovarian cancer cell, a glioblastoma cell, a lung cancer cell such as a non-small cell lung cancer cell, a pancreatic cancer cell). In some aspects, a “target site” can be a tumor target site.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g., a target site such as a cell). In some aspects, the alternating electric field can be in a single direction or multiple directional. In some aspects, alternating electric fields can be delivered through two pairs of transducer arrays that generate perpendicular fields within the target site. For example, for the Optune™ system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and is described in U.S. Pat. No. 7,565,205, which is incorporated herein by reference in its entirety for its teaching of TTFields

In-vivo and in-vitro studies show that the efficacy of TTFields therapy increases as the intensity of the electrical field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g., an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field. For example, the subject of administration can be a subject with brain cancer or breast cancer.

By “treat” is meant to administer or apply a therapeutic, such as alternating electric fields, to a subject, such as a human or other mammal (for example, an animal model), that has cancer or has an increased susceptibility for developing cancer, in order to prevent or delay a worsening of the effects of the disease or infection, or to partially or fully reverse the effects of cancer. For example, treating a subject having glioblastoma can comprise delivering a therapeutic to a cell in the subject.

By “prevent” is meant to minimize or decrease the chance that a subject develops cancer.

As used herein, the terms “administering” and “administration” refer to exposing or applying. Thus, in some aspects, exposing a target site or subject to alternating electric fields or applying alternating electric fields to a target site or subject means administering alternating electric fields to the target site or subject.

As used herein, “organotypic tissue” refers to tissue removed from an organ that can continue to develop as it would if still in that organ.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods of Determining or Testing Efficacy of Alternating Electric Fields

Disclosed are methods of determining the efficacy of an alternating electric field using one or more of three different ex vivo 3D models: microtumors, organoids, tumor slices.

In some aspects, the frequency of the alternating electric fields is between 50 kHz and 1 MHZ, for example between 100 kHz and 500 kHz or between 150 kHz and 300 kHz. In some aspects, the frequency of the alternating electric field is 150 or 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of any of those described herein.

### 1. Use of Microtumors

Disclosed are methods of determining the efficacy of an alternating electric field comprising applying an alternating electric field to one or more microtumors for a period of time, the alternating electric field having a frequency and field strength, wherein the microtumor comprises primary cancer cells; and determining the efficacy of the alternating electric field. In some aspects, the method can be a method of determining the efficacy of an alternating electric field for the treatment of cancer, comprising exposing a microtumor to an alternating electric field; and determining the effect of the alternating electric field on the microtumor.

In some aspects, the microtumors of the disclosed methods display one or more of growth kinetics, active metabolism and a mechanical response to the extra cellular matrix that parallels that of a real tumor. In some aspects, a microtumor is a multi-cellular mass having a diameter of from 30 μm to 150 μm, from 30 μm to 100 μm, from 40 μm to 75 μm, more preferably 50 μm. In some aspects, a microtumor is a multi-cellular mass having a diameter of from 30 μm to 700 μm, from 30 μm to 650 μm, from 30 μm to 600 μm, more preferably 100 μm. In some aspects, a microtumor is a multi-cellular mass having a diameter of 500 μm to 800 μm, 500 μm to 750 μm, 500 μm to 700 μm. In some aspects, a microtumor is a multi-cellular mass having a diameter greater than 1 mm.

In some aspects, the one or more microtumors are formed by seeding primary cancer cells on one or more organotypic tissue slice cultures. In some aspects, the organotypic tissue slice cultures can be from whatever tissue the primary cancer cells are derived from. For example, if the primary cancer cells are glioblastoma cells, then the organotypic tissue slice culture can be an organotypic hippocampal slice culture (OHSC). In another example, the primary cancer cells can be breast cancer cells and the organotypic tissue slice culture can be an organotypic breast tissue slice culture. In some aspects, organotypic tissue slice cultures provide a more naturalistic environment than routine culture of dissociated cells. In some aspects, organotypic tissue slice cultures can be used as described in Schulz et al. Metastasis. Methods in Molecular Biology, vol 2294 pages 59-77, March 2021 hereby incorporated by reference in its entirety as it pertains to the generation and use of OHSCs. In some aspects, the microtumor is formed on an organotypic tissue slice culture that is on a semipermeable membrane. In some aspects, a transparent polyester (PET) membrane can be used. In some aspects, the membrane can be translucent. In some aspects, membranes can be made of polycarbonate or polytetrafluoroethylene (PTFE) material. In some aspects, translucent membranes can be used and can allow for improved TEER measurements or to facilitate better immunofluorescence (IF) microscopy

In some aspects, the microtumor is formed on an organotypic tissue slice culture that is on extra cellular matrix (ECM) protein membranes, including, but not limited to, a collagen membrane, beads (or nanoparticles), hydrogels (e.g. PEGDMA hydrogel), and chips.

In some aspects, the microtumors are not directly cultured on the membrane itself but can be cultured on a brain slice, that can be cultured directly onto the membrane.

In some aspects, the primary cancer cells are patient derived primary cells (PDPCs). The use of PDPCs allows for a direct correlation between the cells used in the disclosed methods (i.e. the PDPCs) and the cells in the subject. For example, the efficacy of the alternating electric fields on the PDPCs used in microtumors can be extrapolated to determine the expected efficacy of the alternating electric fields on a subject, from which the PDPCs were taken. Thus, in some aspects, the disclosed methods can be considered a form of personalized medicine as the treatment of alternating electric fields is tested on PDPCs from a subject and if therapeutic, the alternating electric fields can then be applied to the subject.

In some aspects, the PDPCs are cancer patient derived primary cells. In some aspects, the cancer patient derived primary cells can be from any cancer patient, for example, but not limited to, brain cancer patient, breast cancer patient, lung cancer patient, or liver cancer patient. In some aspects, the brain cancer can be, but is not limited to, glioblastoma (GBM), medulloblastoma, ependymoma, hemangiopericytoma, pincaloblastoma, chordoma, or neuroblastoma. In some aspects, the PDPCs are GBM-patient derived primary cells. In some aspects, the PDPCs are patient derived primary tumor cells. In some aspects, the patient derived primary tumor cells can be from any tumor type. In some aspects, the PDPCs are from lung, pancreatic, hepatocellular or breast cancer cells. In some aspects, the patient derived primary tumor cells can be from any type of tumor derived from brain cancer metastasis.

In some aspects, the PDPCs can be taken from the subject during surgery or biopsy. This can allow for a direct correlation to the efficacy of alternating electric fields on the microtumor formed from the PDPCs to the efficacy of alternating electric fields on the subject from which the PDPCs were derived.

In some aspects, the organotypic tissue slice cultures, such as the OHSCs, can be from any species. In some aspects, the organotypic tissue slice cultures, such as the OHSCs, are murine OHSCs.

In some aspects, the one or more organotypic tissue slice cultures, such as OHSCs, are cultured prior to seeding the one or more organotypic tissue slice cultures with the primary cancer cells. In some aspects, the culturing prior to seeding can be for days or weeks. For example, in some aspects, the one or more OHSCs can be cultured alone for 1-2 weeks prior to seeding the one or more OHSCs with the primary cancer cells.

In some aspects, seeding primary cancer cells on the one or more organotypic tissue slice cultures comprises seeding between 1×10³-1×10⁴ primary cancer cells onto the surface of the one or more organotypic tissue slice cultures. In some aspects, seeding primary cancer cells on the one or more organotypic tissue slice cultures comprises seeding between 1×10³-1×10⁸ primary cancer cells onto the surface of the one or more organotypic tissue slice cultures.

In some aspects, determining the efficacy of the alternating electric field comprises detecting the expression of a marker associated with cell proliferation in the microtumor before applying the alternating electric field and detecting the expression of the marker in the microtumor after applying the alternating electric field, wherein a decrease in expression of the marker in the microtumor indicates the alternating electric field is effective. In some aspects, expression of a marker associated with cell proliferation in the microtumor does not have to be detected before applying the alternating electric field. Rather, in some aspects, a standard can be used for the expression of a marker without exposure to an alternating electric field. For example, standard expression can be established for different microtumors (e.g. GBM, breast, liver, etc). Thus, in some aspects, only detecting the expression of the marker in the microtumor after applying the alternating electric field is required and this can then be compared to a standard expression known for each microtumor type. In some aspects, a marker associated with cell proliferation that decreases in response to an alternating electric field is Ki67.

In some aspects, determining the efficacy of the alternating electric field can be done as stated above. In some aspects, determining the efficacy of the alternating electric field can be determined by determining the size of the microtumor, viability of cells, or total cell count (e.g. wherein viability staining and/or cell count increases). In some aspects, determining the efficacy of the alternating electric field can be determined by determining the expression of particular biomarkers, wherein an increase in expression of a particular marker in the microtumor indicates the alternating electric field is effective. In some aspects, apoptosis markers, such as cleaved Caspase, Akt phosphorylation, p21, p27 and/or gamma-H2AX can be used as biomarkers for determining efficacy, wherein the expression of one or more of these markers is increased after application of the alternating electric field.

In some aspects, determining the efficacy of the alternating electric field comprises detecting apoptosis in the microtumor before applying the alternating electric field and detecting the apoptosis in the microtumor after applying the alternating electric field, wherein an increase in apoptosis in the microtumor indicates the alternating electric field is effective. In some aspects, apoptosis can be measured by, but is not limited to, TUNEL-assay, Annexin V or Cleaved Caspase. In some aspects, determining the efficacy of the alternating electric field can comprise detecting markers for cell cycle and cell cycle arrest. For example, an increase in P21 and P27 can be detected. In some aspects, determining the efficacy of the alternating electric field can comprise detecting an increase in a marker for DNA-double strand breaks, such as, but not limited to gamma-H2AX. In some aspects, determining the efficacy of the alternating electric field can comprise detecting changes in a marker for angiogenesis, such as, but not limited to, CD31.

In some aspects, an increase or decrease in expression or activity can be a 1 fold, 1.5 fold, 2 fold, or higher increase or decrease compared to the before alternating electric field numbers. In some aspects, an increase or decrease in expression or activity can be a significant increase or decrease compared to the before alternating electric field numbers using statistical significance (e.g. p-value below or equal to 0.05).

In some aspects, determining the efficacy of the alternating electric field comprises measuring a size of the microtumor prior to and after applying the alternating electrical field, wherein a decrease in the size of the microtumor after applying the alternating electrical field indicates the alternating electric field is effective. In some aspects, the decrease can be 10% or higher compared to the before alternating electric field numbers. In some aspects, the decrease can be a significant decrease compared to the before alternating electric field numbers using statistical significance (e.g. p-value below or equal to 0.05).

In some aspects, determining the efficacy of alternating electric fields comprises using any known method in the art for testing efficacy of a treatment.

In some aspects, the disclosed methods of determining efficacy of alternating electric fields can further comprise a treatment step to a subject once it has been determined that alternating electric fields would be an effective treatment. The disclosed methods of determining efficacy of alternating electric fields comprises an ex vivo method that can be personalized and result in administration of alternating electric fields to the subject determined to be a good recipient of alternating electric fields (e.g. good efficacy of alternating electric fields in the ex vivo model). Thus, the disclosed methods further comprise applying alternating electric fields to a target site of a subject for a period of time, the alternating electric fields having a frequency and field strength, wherein the primary cancer cells are derived from the subject, wherein the target site comprises cancer cells.

In other embodiments the disclosed methods can result in administration of alternating electric fields in combination with administration of a sensitizing agent to the subject determined to not be a good recipient of alternating electric fields alone (e.g. weak efficacy of alternating electric fields alone in the ex vivo model). Examples of suitable sensitizing agents include those described in U.S. Patent Application Publication Nos. 2020/0306531, 2021/0038584, and 2022/0096818, each of which are hereby incorporated by reference in their entirety for their teaching of suitable sensitizing agents.

In some aspects, the disclosed methods further comprise obtaining the primary cancer cells from a subject prior to forming the microtumor.

### 2. Use of Organoids

Disclosed are methods of testing the efficacy of an alternating electric field comprising applying alternating electric fields to one or more organoids for a period of time, the alternating electric fields having a frequency and field strength, wherein the organoids are cultured on organotypic hippocampal slice cultures; and determining the efficacy of alternating electric fields. In some aspects, organoids maintain the tumor-inherent invasive, immunohistological, cellular, and mutational profile of the tumor from which they are derived but not the tumor microenvironment or histopathological tissue structure.

In some aspects, one or more organoids can be generated by processing tumor tissue from a patient and cutting into small pieces. In some aspects, the tissue pieces are about 200 to 600 μm. In some aspects, the tissue pieces are about 500 μm. The tissue can then be cultured, or grown, to form one or more organoids. Once organoids have formed, the organoids can then be cultured or incubated on organotypic tissue slice cultures. As an example, fresh intraoperatively obtained tumor tissue can be stored on ice in Hibernate A medium (Gibco, Carlsbad, USA). Next, the tissue can be cleared of necrosis and blood vessels and carefully minced with a scalpel into approximately 0.5 mm large pieces under the microscope. These pieces can then be treated with RBC Lysis Buffer (Invitrogen, Carlsbad, USA) for 10 min and washed two times with Hibernate A medium containing 1% Glutamax, 0.4% penicillin/streptomycin and 0.1% Amphotericin (HGPSA) (all from Gibco, Carlsbad, USA). Sections can be transferred into GBO medium consisting of 47.24% DMEM/F12, 47.25% Neurobasal, 0.02% B27 without Vitamin A (50x), 0.01% Glutamax, 0.01% N2, 0.01% NEAA, 0.004% penicillin/streptomycin, 0.001% B-Mercaptoethanol (all from Gibco, Carlsbad, USA) and 0.00023% human insulin (Sigma-Aldrich, St. Louis, USA) to ultra-low attachment 6-well plates (Corning Costar, New York, USA) and incubated at 37° C., 5% CO2 and 95% humidity on an orbital shaker at 120 rpm. After 2 weeks of culture, organoids can form.

In some aspects, the organotypic tissue slice cultures can be from whatever tissue the organoids are derived from. For example, if the organoids are derived from glioblastoma cells, then the organotypic tissue slice culture can be OHSC as both the organoid and the organotypic tissue slice culture are derived from brain tissue. In another example, the organoid can be derived from breast cancer cells and the organotypic tissue slice culture can be an organotypic breast tissue slice culture. In some aspects, organotypic tissue slice cultures provide a more naturalistic environment than routine culture of dissociated cells.

In some aspects, the organoids are subject derived and generated from tumor tissue from the subject. In some aspects, the tumor tissue is taken from the subject during surgery or biopsy. Thus, there is a direct correlation to the efficacy of alternating electric fields on the organoid to the efficacy of alternating electric fields on the subject from which the organoids were derived.

In some aspects, the culturing or incubating of the one or more organoids on organotypic tissue slice cultures is conducted on a semipermeable membrane. In some aspects, a transparent polyester (PET) membrane can be used. In some aspects, the membrane can be translucent. And membranes could also be made up of polycarbonate or polytetrafluoroethylene (PTFE) material. Translucent is better for TEER measurements, while transparent membranes facilitate better IF microscopy.

In some aspects, the organoid is formed on an organotypic tissue slice culture that is on extra cellular matrix (ECM) protein membranes, such as but not limited to a collagen membrane, beads (or nanoparticles), hydrogels, for example, PEGDMA hydrogel, and chips.

In some aspects, the one or more organotypic tissue slice cultures, such as OHSCs, are cultured prior to culturing/incubating with the organoids. In some aspects, the culturing prior to incubating with organoids can be for days or weeks. For example, in some aspects, the one or more OHSCs can be cultured alone for 1-2 weeks prior to culturing/incubating the one or more OHSCs with the one or more organoids.

In some aspects, the organoid is between 100 and 1000 μm. In some aspects, the organoid is between 100 and 600 μm. In some aspects, the organoid is between 200 and 800 μm. In some aspects, the organoid is between 300 and 700 μm. In some aspects, the organoid is between 500 and 600 μm.

In some aspects, determining the efficacy of the alternating electric field comprises detecting the expression of a marker associated with cell proliferation in the organoid before applying the alternating electric field and detecting the expression of the marker in the organoid after applying the alternating electric field, wherein a decrease in expression of the marker in the organoid indicates the alternating electric field is effective. In some aspects, expression of a marker associated with cell proliferation in the organoid does not have to be detected before applying the alternating electric field. Rather, in some aspects, a standard can be used for the expression of a marker without exposure to an alternating electric field. For example, standard expression can be established for different organoids (e.g. GBM, breast, liver, etc.). Thus, in some aspects, only detecting the expression of the marker in the organoid after applying the alternating electric field is required and this can then be compared to a standard expression known for each organoid type. In some aspects, a marker associated with cell proliferation that decreases in response to an alternating electric field is Ki67.

In some aspects, determining the efficacy of the alternating electric field can be done as stated above except an increase in expression of the marker in the organoid indicates the alternating electric field is effective. In some aspects, apoptosis markers, such as cleaved Caspase, Akt phosphorylation, p21, p27 and/or gamma-H2AX are increased indicating the efficacy of the alternating electric field.

In some aspects, determining the efficacy of the alternating electric field comprises detecting apoptosis in the organoid before applying the alternating electric field and detecting the apoptosis in the organoid after applying the alternating electric field, wherein a increase in apoptosis in the organoid indicates the alternating electric field is effective. In some aspects, apoptosis can be measured by, but is not limited to, TUNEL-assay, Annexin V or Cleaved Caspase. In some aspects, determining the efficacy of the alternating electric field can comprise detecting markers for cell cycle and cell cycle arrest. For example, an increase in P21 and P27 can be detected. In some aspects, determining the efficacy of the alternating electric field can comprise detecting an increase in a marker for DNA-double strand breaks, such as, but not limited to gamma-H2AX. In some aspects, determining the efficacy of the alternating electric field can comprise detecting changes in a marker for angiogenesis, such as, but not limited to, CD31.

In some aspects, an increase or decrease in expression or activity can be a 1 fold, 1.5 fold, 2 fold, or higher increase or decrease compared to the before alternating electric field numbers. In some aspects, an increase or decrease in expression or activity can be a significant increase or decrease compared to the before alternating electric field numbers using statistical significance (e.g. p-value below or equal to 0.05).

In some aspects, determining the efficacy of the alternating electric field comprises measuring a size of the organoid prior to and after applying the alternating electrical field, wherein a decrease in the size of the organoid after applying the alternating electrical field indicates the alternating electric field is effective. In some aspects, the decrease can be 10% or higher compared to the before alternating electric field numbers. In some aspects, the decrease can be a significant decrease compared to the before alternating electric field numbers using statistical significance (e.g. p-value below or equal to 0.05).

In some aspects, determining the efficacy of alternating electric fields comprises using any known method in the art for testing efficacy of a treatment.

In some aspects, the disclosed methods of determining efficacy of alternating electric fields can further comprise a treatment step to a subject once it has been determined that alternating electric fields would be an effective treatment. The disclosed methods of determining efficacy of alternating electric fields comprises an ex vivo method that can be personalized and result in administration of alternating electric fields to the subject determined to be a good recipient of alternating electric fields (e.g. good efficacy of alternating electric fields in the ex vivo model). Thus, the disclosed methods further comprise applying alternating electric fields to a target site of a subject for a period of time, the alternating electric fields having a frequency and field strength, wherein the organoids are derived from the subject, wherein the target site comprises cancer cells.

In some aspects, organoids derived from a subject include organoids made of tissue pieces derived from the subject. In some aspects, one or more organoids can be generated by processing tumor tissue from a patient and cutting into small pieces. In some aspects, the tissue pieces are about 200 to 600 μm. In some aspects, the tissue pieces are about 500 μm. The tissue can then be cultured, or grown, to form organoids. Once organoids have formed, the organoids can then be cultured or incubated on organotypic tissue slice cultures. As an example, fresh intraoperatively obtained tumor tissue can be stored on ice in Hibernate A medium (Gibco, Carlsbad, USA). The tissue can then be cleared of necrosis and blood vessels and carefully minced with a scalpel into approximately 0.5 mm large pieces under the microscope. These pieces can then be treated with RBC Lysis Buffer (Invitrogen, Carlsbad, USA) for 10 min and washed two times with Hibernate A medium containing 1% Glutamax, 0.4% penicillin/streptomycin and 0.1% Amphotericin (HGPSA) (all from Gibco, Carlsbad, USA). Sections can be transferred into GBO medium consisting of 47.24% DMEM/F12, 47.25% Neurobasal, 0.02% B27 without Vitamin A (50x), 0.01% Glutamax, 0.01% N2, 0.01% NEAA, 0.004% penicillin/streptomycin, 0.001% B-Mercaptoethanol (all from Gibco, Carlsbad, USA) and 0.00023% human insulin (Sigma-Aldrich, St. Louis, USA) to ultra-low attachment 6-well plates (Corning Costar, New York, USA) and incubated at 37° C., 5% CO₂ and 95% humidity on an orbital shaker at 120 rpm. After 2 weeks of culture, organoids can form.

In other embodiments the disclosed methods can result in administration of alternating electric fields in combination with administration of a sensitizing agent to the subject determined to not be a good recipient of alternating electric fields alone (e.g. weak efficacy of alternating electric fields alone in the ex vivo model). Examples of suitable sensitizing agents include those described in U.S. Patent Application Publication Nos. 2020/0306531, 2021/0038584, and 2022/0096818, each of which are hereby incorporated by reference in their entirety for teaching suitable sensitizing agents.

In some aspects, the disclosed methods further comprise obtaining a tumor tissue or biopsy from a subject and producing an organoid for use in the methods disclosed herein.

### 3. Use of Tumor Slices

Disclosed are methods of testing the efficacy of alternating electric fields on a subject comprising culturing or incubating one or more tumor slices from the subject, applying alternating electric fields to the one or more tumor slices for a period of time, the alternating electric fields having a frequency and field strength, and determining the efficacy of alternating electric fields. In some aspects, unlike organoids, tumor slices maintain the tumor-inherent invasive, immunohistological, cellular, and mutational profile of the tumor from which they are derived and the tumor microenvironment or histopathological tissue structure.

In some aspects, culturing or incubating the one or more tumor slices occurs on a semipermeable membrane. In some aspects, a transparent polyester (PET) membrane can be used. In some aspects, the membrane can be translucent. In some aspects, membranes can be made of polycarbonate or polytetrafluoroethylene (PTFE) material. In some aspects, translucent membranes can be used and can allow for improved TEER measurements or to facilitate better IF microscopy.

In some aspects, determining the efficacy of the alternating electric field comprises detecting the expression of a marker associated with cell proliferation in the tumor slices before applying the alternating electric field and detecting the expression of the marker in the tumor slices after applying the alternating electric field, wherein a decrease in expression of the marker in the tumor slices indicates the alternating electric field is effective. In some aspects, expression of a marker associated with cell proliferation in the tumor slices does not have to be detected before applying the alternating electric field. Rather, in some aspects, a standard can be used for the expression of a marker without exposure to an alternating electric field. For example, standard expression can be established for different tumor slices (e.g. GBM, breast, liver, etc). Thus, in some aspects, only detecting the expression of the marker in the tumor slices after applying the alternating electric field is required and this can then be compared to a standard expression known for each tumor slice type. In some aspects, a marker associated with cell proliferation that decreases in response to an alternating electric field is Ki67.

In some aspects, determining the efficacy of the alternating electric field can be done as stated above except an increase in expression of the marker in the tumor slices indicates the alternating electric field is effective. In some aspects, apoptosis markers, such as cleaved Caspase, Akt phosphorylation, p21, p27 and/or gamma-H2AX can be used as biomarkers for determining efficacy, wherein the expression of one or more of these markers is increased after application of the alternating electric field.

In some aspects, determining the efficacy of the alternating electric field comprises detecting apoptosis in the tumor slices before applying the alternating electric field and detecting the apoptosis in the tumor slices after applying the alternating electric field, wherein an increase in apoptosis in the tumor slices indicates the alternating electric field is effective. In some aspects, apoptosis can be measured by, but is not limited to, TUNEL-assay, Annexin V or Cleaved Caspase. In some aspects, determining the efficacy of the alternating electric field can comprise detecting markers for cell cycle and cell cycle arrest. For example, an increase in P21 and P27 can be detected. In some aspects, determining the efficacy of the alternating electric field can comprise detecting an increase in a marker for DNA-double strand breaks, such as, but not limited to gamma-H2AX. In some aspects, determining the efficacy of the alternating electric field can comprise detecting a change in a marker for angiogenesis, such as, but not limited to, CD31.

In some aspects, an increase or decrease in expression or activity can be a 1 fold, 1.5 fold, 2 fold, or higher increase or decrease compared to the before alternating electric field numbers. In some aspects, an increase or decrease in expression or activity can be a significant increase or decrease compared to the before alternating electric field numbers using statistical significance (e.g. p-value below or equal to 0.05).

In some aspects, determining the efficacy of alternating electric fields comprises using any known method in the art for testing efficacy of a treatment.

In some aspects, the disclosed methods of determining efficacy of alternating electric fields can further comprise a treatment step to a subject once it has been determined that alternating electric fields would be an effective treatment. The disclosed methods of determining efficacy of alternating electric fields comprises an ex vivo method that can be personalized and result in administration of alternating electric fields to the subject determined to be a good recipient of alternating electric fields (e.g. good efficacy of alternating electric fields in the ex vivo model). Thus, the disclosed methods further comprise applying alternating electric fields to a target site of a subject for a period of time, the alternating electric fields having a frequency and field strength, wherein the tumor slices are derived from the subject, wherein the target site comprises cancer cells.

In other embodiments the disclosed methods can result in administration of alternating electric fields in combination with administration of a sensitizing agent to the subject determined to not be a good recipient of alternating electric fields alone (e.g. weak efficacy of alternating electric fields alone in the ex vivo model). Examples of suitable sensitizing agents include those described in U.S. Patent Application Publication Nos. 2020/0306531, 2021/0038584, and 2022/0096818, each of which are hereby incorporated by reference in its entirety.

In some aspects, the disclosed methods further comprise obtaining a tumor slice from a subject. In some aspects, the tumor slice can be prepared from a biopsy obtained from a subject or a tumor sample obtained from the subject (e.g. via surgery).

### C. Methods of Determining Parameters for Alternating Electric Fields

Disclosed are methods of using any of the disclosed ex vivo models of microtumors, organoids or tumor slices to determine what parameters of an alternating electric field would be best for treating a subject from which the models of microtumors, organoids or tumor slices originated.

Disclosed are methods of determining or selecting a value of a parameter of an alternating electric field to be administered for treating a disease or condition, the method comprising applying an alternating electric field to at least two microtumors produced from cells obtained from a subject, wherein the alternating electric field applied to each of the at least two microtumors has a different condition (e.g. frequency, field strength) from the other, and determining which condition is most effective at reducing viability of cancer cells in the microtumors. In some aspects, the methods can further comprise treating the subject, from which the microtumors were derived, with an alternating electric field having the determined frequency (e.g., frequency that resulted in higher reduced viability of cancers cells in the microtumors).

Disclosed are methods of determining or selecting a value of a parameter of an alternating electric field to be administered for treating a disease or condition, the method comprising applying an alternating electric field to at least two organoids produced from tissue obtained from a subject, wherein the alternating electric field applied to each of the at least two organoids has a different condition (e.g. frequency, field strength) from the other, and determining which condition (e.g. frequency, field strength) is most effective at reducing viability of cancer cells in the organoids. In some aspects, the methods can further comprise treating the subject, from which the organoids, or tissue used to produce the organoids, were derived, with an alternating electric field having the determined frequency (e.g., frequency that resulted in higher reduced viability of cancers cells in the organoids).

Disclosed are methods of determining or selecting a value of a parameter of an alternating electric field to be administered for treating a disease or condition, the method comprising applying an alternating electric field to at least two of a plurality of tumor slices produced from cells obtained from a subject, wherein the alternating electric field applied to each of the at least two tumor slices has a different condition (e.g. frequency, field strength) from the other, and determining which condition (e.g. frequency, field strength) is most effective at reducing viability of cancer cells in the tumor slices. In some aspects, the methods can further comprise treating the subject, from which the tumor slices were derived, with an alternating electric field having the determined frequency (e.g., frequency that resulted in higher reduced viability of cancers cells in the tumor slices).

In some aspects, other parameters of the alternating electric fields can be determined. For example, the best period of time for applying the alternating electric fields can be determined or the best field strength for the alternating electric fields can be determined. In some aspects, combinations of these parameters can be determined. For example, microtumors, organoids and/or tumor slices can be exposed to alternating electric fields having different frequencies, times of applying, and/or field strength and the best combination can be determined.

In some aspects, determining which parameter has the best efficacy can be achieved by using similar methods as described above. For example, determining the efficacy of the alternating electric field comprises detecting the expression of a marker associated with cell proliferation in the microtumor, organoid, or tumor slice before applying the alternating electric field and detecting the expression of the marker in the microtumor, organoid, or tumor slice after applying the alternating electric field, wherein a decrease in expression of the marker in the microtumor, organoid, or tumor slice indicates the alternating electric field is effective. In some aspects, expression of a marker associated with cell proliferation in the microtumor, organoid, or tumor slice does not have to be detected before applying the alternating electric field. Rather, in some aspects, a standard can be used for the expression of a marker without exposure to an alternating electric field. For example, standard expression can be established for different microtumors, organoids, or tumor slices (e.g., GBM, breast, liver, etc). Thus, in some aspects, only detecting the expression of the marker in the microtumor, organoid, or tumor slice after applying the alternating electric field is required and this can then be compared to a standard expression known for each microtumor, organoid, or tumor slice type. In some aspects, a marker associated with cell proliferation that decreases in response to an alternating electric field is Ki67.

In some aspects, determining the efficacy of the alternating electric field can be done as stated above except an increase in expression of the marker in the microtumor, organoid, or tumor slice indicates the alternating electric field is effective. In some aspects, apoptosis markers, such as cleaved Caspase, Akt phosphorylation, p21, p27 and/or gamma-H2AX can be used as biomarkers for determining efficacy, wherein the expression of one or more of these markers is increased after application of the alternating electric field.

In some aspects, determining the efficacy of the alternating electric field comprises detecting apoptosis in the microtumor, organoid, or tumor slice before applying the alternating electric field and detecting the apoptosis in the microtumor, organoid, or tumor slice after applying the alternating electric field, wherein an increase in apoptosis in the organoid indicates the alternating electric field is effective. In some aspects, apoptosis can be measured by, but is not limited to, TUNEL-assay, Annexin V or Cleaved Caspase. In some aspects, determining the efficacy of the alternating electric field can comprise detecting markers for cell cycle and cell cycle arrest. For example, an increase in P21 and P27 can be detected. In some aspects, determining the efficacy of the alternating electric field can comprise detecting an increase in a marker for DNA-double strand breaks, such as, but not limited to gamma-H2AX. In some aspects, determining the efficacy of the alternating electric field can comprise detecting a change in a marker for angiogenesis, such as, but not limited to, CD31.

In some aspects, an increase or decrease in expression or activity can be a 1 fold, 1.5 fold, 2 fold, or higher increase or decrease compared to the before alternating electric field numbers. In some aspects, an increase or decrease in expression or activity can be a significant increase or decrease compared to the before alternating electric field numbers using statistical significance (e.g. p-value below or equal to 0.05).

In some aspects, determining the efficacy of the alternating electric field comprises measuring a size of the microtumor or organoid prior to and after applying the alternating electrical field, wherein a decrease in the size of the microtumor or organoid after applying the alternating electrical field indicates the alternating electric field is effective. In some aspects, the decrease can be 10% or higher compared to the before applying alternating electric field measurement. In some aspects, the decrease can be a significant decrease compared to the before alternating electric field numbers using statistical significance (e.g. p-value below or equal to 0.05).

In some aspects, determining the efficacy of alternating electric fields comprises using any known method in the art for testing efficacy of a treatment.

In some aspects, the periods of time for applying alternating electric fields can be, but are not limited to, about 2, 4, 6, 8, 12, 24, 36, 48, 60, 72, 84, 96, 112, 124, 136, 148, or 168 hours. In some aspects, the frequency of the alternating electric fields can be, but is not limited to, between 50 kHz and 1 MHZ, for example between 100 kHz and 500 kHz, or between 150 kHz and 300 kHz. In some aspects, the field strength can be, but is not limited to, between 0.1 V/cm and 10 V/cm, for example between 0.5V/cm and 2V/cm.

### D. Methods of Treating

Disclosed are methods of treating a subject having cancer comprising determining the efficacy of alternating electric fields in an ex vivo model derived from the subject, and applying alternating electric fields to the subject when the alternating electric fields are determined to be effective in the ex vivo model.

Disclosed are methods of treating a subject having cancer comprising determining the parameters of an alternating electric field effective for treating the cancer in the subject, and applying the alternating electric fields, using the parameters determined to be effective, to a target site of the subject, wherein the target site comprises one or more cancer cells.

In some aspects, the alternating electric fields are applied for a period of time and the alternating electric fields have a frequency and field strength.

In some aspects, determining the efficacy of alternating electric fields or determining the parameters of an alternating electric field effective for treating a subject having cancer can be achieved using similar methods described herein. For example, in some aspects, determining which parameter has the best efficacy can be achieved by using similar methods as described above. For example, determining the efficacy of the alternating electric field comprises detecting the expression of a marker associated with cell proliferation in a microtumor, organoid, or tumor slice before applying the alternating electric field and detecting the expression of the marker in the microtumor, organoid, or tumor slice after applying the alternating electric field, wherein a decrease in expression of the marker in the organoid indicates the alternating electric field is effective. In some aspects, expression of a marker associated with cell proliferation in a microtumor, organoid, or tumor slice does not have to be detected before applying the alternating electric field. Rather, in some aspects, a standard can be used for the expression of a marker without exposure to an alternating electric field. For example, standard expression can be established for different microtumors, organoids, or tumor slices (e.g. GBM, breast, liver, etc). Thus, in some aspects, only detecting the expression of the marker in the microtumor, organoid, or tumor slice after applying the alternating electric field is required and this can then be compared to a standard expression known for each microtumor, organoid, or tumor slice type. In some aspects, a marker associated with cell proliferation that decreases in response to an alternating electric field is Ki67.

In some aspects, determining the efficacy of the alternating electric field comprises detecting apoptosis in the microtumor, organoid, or tumor slice before applying the alternating electric field and detecting the apoptosis in the microtumor, organoid, or tumor slice after applying the alternating electric field, wherein an increase in apoptosis in the organoid indicates the alternating electric field is effective. In some aspects, apoptosis can be measured by, but is not limited to, TUNEL-assay, Annexin V or Cleaved Caspase. In some aspects, determining the efficacy of the alternating electric field can comprise detecting markers for cell cycle and cell cycle arrest. For example, an increase in P21 and P27 can be detected. In some aspects, determining the efficacy of the alternating electric field can comprise detecting an increase in a marker for DNA-double strand breaks, such as, but not limited to gamma-H2AX. In some aspects, determining the efficacy of the alternating electric field can comprise detecting a change in a marker for angiogenesis, such as, but not limited to, CD31.

In some aspects, determining the efficacy of the alternating electric field can be done as stated above except an increase in expression of the marker in the microtumor, organoid, or tumor slice indicates the alternating electric field is effective. In some aspects, apoptosis markers, such as cleaved Caspase, Akt phosphorylation, p21, p27 and/or gamma-H2AX can be used as biomarkers for determining efficacy, wherein the expression of one or more of these markers is increased after application of the alternating electric field.

In some aspects, an increase or decrease in expression or activity can be a 1 fold, 1.5 fold, 2 fold, or higher increase or decrease compared to the before alternating electric field numbers. In some aspects, an increase or decrease in expression or activity can be a significant increase or decrease compared to the before alternating electric field numbers using statistical significance (e.g. p-value below or equal to 0.05).

In some aspects, determining the efficacy of the alternating electric field comprises measuring a size of the microtumor or organoid prior to and after applying the alternating electrical field, wherein a decrease in the size of the microtumor or organoid after applying the alternating electrical field indicates the alternating electric field is effective. In some aspects, the decrease can be 10% or higher compared to the before applying alternating electric field measurement. In some aspects, the decrease can be a significant decrease compared to the before alternating electric field numbers using statistical significance (e.g. p-value below or equal to 0.05).

In some aspects, determining the efficacy of alternating electric fields comprises using any known method in the art for testing efficacy of a treatment.

In some embodiments the disclosed methods can result in administration of alternating electric fields in combination with administration of a sensitizing agent to the subject determined to not be a good recipient of alternating electric fields alone (e.g. weak efficacy of alternating electric fields alone in the ex vivo model). Examples of suitable sensitizing agents include those described in U.S. Patent Application Publication Nos. 2020/0306531, 2021/0038584, and 2022/0096818, each of which are hereby incorporated by reference in its entirety.

In some aspects, the disclosed methods further comprise obtaining one or more of patient derived primary cells, tumor pieces, and/or tumor slice from the subject.

### E. Methods of Identifying Biomarkers

Disclosed are methods of identifying biomarkers that respond to application of an alternating electric field comprising performing a morphological or molecular analysis of a microtumor formed from primary cancer cells obtained from a subject, an organoid derived from tissue from a subject, or a tumor slice from a subject; applying an alternating electric field to the microtumor, organoid or tumor slice for a period of time, the alternating electric field having a frequency and field strength; performing a morphological or molecular analysis of the microtumor, organoid or tumor slice after the step of applying an alternating electric field to the microtumor, organoid or tumor slice; and comparing the morphological or molecular analysis of performing a morphological or molecular analysis of the microtumor, organoid or tumor slice prior to applying alternating electric fields with the morphological or molecular analysis performed after applying alternating electric fields, wherein a difference in the morphological or molecular analysis of before applying alternating electric fields with the morphological or molecular analysis of after applying alternating electric fields identifies a biomarker that responds to application of an alternating electric field.

In some aspects, the step of obtaining primary cancer cells from a subject and forming a microtumor from the primary cancer cells, obtaining organoids/tissue from a subject and forming larger organoids, or obtaining a tumor slice from a subject can be performed prior to performing a morphological or molecular analysis of the microtumor, organoid or tumor slice.

In some aspects, performing a morphological or molecular analysis of the microtumor, organoid or tumor slice comprises detecting the expression of a marker associated with cell proliferation in the microtumor, organoid, or tumor slice before applying the alternating electric field and detecting the expression of the marker in the microtumor, organoid, or tumor slice after applying the alternating electric field, wherein a decrease in expression of the marker in the microtumor, organoid, or tumor slice indicates a difference in the molecular analysis and thus identifies a biomarker that responds to application of an alternating electric field. In some aspects, expression of a marker associated with cell proliferation in the microtumor, organoid, or tumor slice does not have to be detected before applying the alternating electric field. Rather, in some aspects, a standard can be used for the expression of a marker without exposure to an alternating electric field. For example, standard expression can be established for different microtumors, organoids, or tumor slices (e.g. GBM, breast, liver, etc). Thus, in some aspects, only detecting the expression of the marker in the microtumor, organoid, or tumor slice after applying the alternating electric field is required and this can then be compared to a standard expression known for each microtumor, organoid, or tumor slice type. In some aspects, a marker associated with cell proliferation that decreases in response to an alternating electric field is Ki67.

In some aspects, performing a morphological or molecular analysis can be done as stated above except an increase in expression of the marker in the microtumor, organoid, or tumor slice indicates the alternating electric field is effective. In some aspects, apoptosis markers, such as cleaved Caspase, Akt phosphorylation, p21, p27 and/or gamma-H2AX can be used as biomarkers for determining efficacy, wherein the expression of one or more of these markers is increased after application of the alternating electric field.

In some aspects, performing a morphological or molecular analysis of the microtumor, organoid or tumor slice comprises detecting apoptosis in the microtumor, organoid, or organotypic tumor slice before applying the alternating electric field and detecting the apoptosis in the microtumor, organoid, or tumor slice after applying the alternating electric field, wherein an increase in apoptosis of the marker in the microtumor, organoid, or tumor slice indicates a difference in the morphological molecular analysis and thus identifies a biomarker that responds to application of an alternating electric field. In some aspects, apoptosis can be measured by, but is not limited to, TUNEL-assay, Annexin V or Cleaved Caspase. In some aspects, determining the efficacy of the alternating electric field can comprise detecting markers for cell cycle and cell cycle arrest. For example, an increase in P21 and P27 can be detected. In some aspects, performing a morphological or molecular analysis of the microtumor, organoid or tumor slice comprises detecting an increase in a marker for DNA-double strand breaks, such as, but not limited to gamma-H2AX. In some aspects, performing a morphological or molecular analysis of the microtumor, organoid or tumor slice comprises detecting a change in a marker for angiogenesis, such as, but not limited to, CD31.

In some aspects, an increase or decrease in expression or activity can be a 1 fold, 1.5 fold, 2 fold, or higher increase or decrease compared to the before alternating electric field numbers. In some aspects, an increase or decrease in expression or activity can be a significant increase or decrease compared to the before alternating electric field numbers using statistical significance (e.g. p-value below or equal to 0.05).

In some aspects, performing a morphological or molecular analysis of the microtumor or organoid comprises measuring a size of the microtumor or organoid prior to and after applying the alternating electrical field, wherein a decrease in the size of the microtumor or organoid after applying the alternating electrical field indicates the alternating electric field is effective. In some aspects, the decrease can be 10% or higher compared to the before alternating electric field measurement. In some aspects, the decrease can be a significant decrease compared to the before alternating electric field numbers using statistical significance (e.g. p-value below or equal to 0.05).

In some aspects, determining the efficacy of alternating electric fields comprises using any known method in the art for testing efficacy of a treatment.

In some aspects, the disclosed methods of identifying biomarkers comprise testing for an increase or decrease in ER (estrogen receptor), HER/neu-2, EGFR, KRAS, Akt, PI3KA, BRAF, ALK, ROS, PD-1, PD-L1, RET, LAG-3, Ox40, TIGIT, TIM3, VISTA, Foxp3, CD33, CD14, CD15 or IDO, TCR or interferon gamma after the application of an alternating electric field. These are known biomarkers for different cancers that are commonly tested for in biopsies and therefore can be used in the current methods.

### F. Alternating Electric Fields

The methods disclosed herein comprise alternating electric fields. In some aspects, the alternating electric field used in the methods disclosed herein is a tumor-treating field (TTField). In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the Inovitro™ laboratory research system (Novocure, Haifa, Israel) can be used for TTFields administration. U87MG cells and PDPC cultured as monolayers can be treated by using glass coverslips with 20 mm diameter (Hartenstein, Würzburg, Germany) placed into Inovitro™ ceramic dishes (Novocure, Haifa, Israel). Cells can be trypsinized and plated onto the coverslips by placing 350 μl cell culture medium containing 30,000 cells as a drop in their center. Cells can attach during incubation (e.g. 20 h incubation at 37° C. and 5% CO₂). The medium can be replaced by 2 ml fresh cell culture medium, the ceramic dishes can be placed onto a base plate connected to a TTFields generator and TTFields at 200 kHz can be applied with an intensity of 1.7 V/cm for 72 h. The medium can be renewed every 48 h. Control cells can be kept under the same conditions without TTFields application. To evaluate TTFields effects, cells can be trypsinized and counted using the Scepter 2.1 cell counter (Merck, Darmstadt, Germany).

To treat OHSC and tumor slices with TTFields, ceramic dishes with high walls (Novocure, Haifa, Israel) can be utilized. The holders containing inserts with semipermeable membranes can be placed into the dishes and 2.5 ml brain slice medium can be pipetted into the dishes outside the inserts. OHSC and tumor slices, respectively, can be transferred as described above. To avoid condensation water to drop onto the slice surfaces, a 12 mm coverslip was placed onto each insert. The ceramic dish can be covered with parafilm and closed with a lid, to minimize evaporation of the medium. TTFields can be applied for 72 to 96 h at 200 kHz and 1.5 V/cm. Condensation water can be carefully aspirated every day, the medium changed every second day.

In some aspects, the frequency of the alternating electric field is between 50 kHz and 1 MHz. In some aspects, the frequency of the alternating electric field is between 100 and 500 kHz. The frequency of the alternating electric fields can also be, but is not limited to, between 50 and 500 kHz, between 100 and 500 kHz, between 25 kHz and 1 MHZ, between 50 and 190 kHz, between 25 and 190 kHz, between 180 and 220 kHz, or between 210 and 400 kHz. In some aspects, the frequency of the alternating electric fields can be electric fields at 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 KHz.

In some aspects, the field strength of the alternating electric fields can be between 0.1 and 10 V/cm RMS, for example between 0.5 and 4 V/cm RMS. In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm RMS. In some aspects, the field strength can be 0.9 V/cm RMS. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours, 20 minutes to 168 hours, or any time between. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more. In some aspects the exposure may last for at least 6 hours, at least 12 hours, at least 18 hours, or at least 19 hours per 24 hour period over the course of one or more (e.g., three or four) days. In some aspects the alternating electric fields are applied for at least 20%, at least 40%, at least 50%, at least 60%, at least 70%, or at least 80% of the time over a period of three days, four days, one week, one month, three months, or longer than three months.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

### G. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits comprising tumor slice cultures and one or more materials for delivering alternating electric fields, such as the OPTUNE® system.

### H. Embodiments

Embodiment 1 discloses a method of determining the efficacy of an alternating electric field comprising applying an alternating electric field to a microtumor for a period of time, the alternating electric field having a frequency and field strength, wherein the microtumor comprises primary cancer cells; and determining the efficacy of the alternating electric field.

Embodiment 2 discloses embodiment 1, wherein the microtumor is formed by seeding primary cancer cells on an organotypic tissue slice culture.

Embodiment 3 discloses embodiment 2, wherein the organotypic tissue slice culture is an organotypic hippocampal slice culture (OHSC).

Embodiment 4 discloses embodiments 1-3, wherein the primary cancer cells are patient derived primary cells (PDPC).

Embodiment 5 discloses embodiment 4, wherein the PDPCs are GBM-patient derived primary cells.

Embodiment 6 discloses embodiments 3-5, wherein the OHSC is murine OHSC.

Embodiment 7 discloses embodiments 2-6, wherein the organotypic tissue slice culture is cultured alone for 1-2 weeks prior to seeding the organotypic tissue slice culture with the primary cancer cells.

Embodiment 8 discloses embodiments 2-7, wherein seeding primary cancer cells an organotypic tissue slice culture comprises seeding between 1×10³-1×10⁴ primary cancer cells onto the surface of the organotypic tissue slice culture.

Embodiment 9 discloses embodiments 1-8, wherein the microtumor is formed on an organotypic tissue slice culture on a semipermeable membrane.

Embodiment 10 discloses embodiments 1-9, wherein determining the efficacy of the alternating electric field comprises detecting the expression of a marker associated with cell proliferation in the microtumor before applying the alternating electric field and detecting the expression of the marker in the microtumor after applying the alternating electric field, wherein a decrease in expression of the marker in the microtumor indicates the alternating electric field is effective.

Embodiment 11 discloses embodiments 1-10, wherein determining the efficacy of the alternating electric field comprises measuring a size of the microtumor prior to and after applying the alternating electrical field, wherein a decrease in the size of the microtumor after applying the alternating electrical field indicates the alternating electric field is effective.

Embodiment 12 discloses embodiments 2-11, further comprising applying alternating electric fields to a target site of a subject for a period of time, the alternating electric fields having a frequency and field strength, wherein the primary cancer cells are derived from the subject, wherein the target site comprises cancer cells.

Embodiment 13 discloses a method of determining the efficacy of an alternating electric field comprising: applying alternating electric fields to an organoid for a period of time, the alternating electric fields having a frequency and field strength; and determining the efficacy of alternating electric fields.

Embodiment 14 discloses embodiment 13, wherein the organoid is cultured on an organotypic tissue slice culture before applying alternating electric fields.

Embodiment 15 discloses embodiment 14, wherein the organotypic tissue slice culture is an OHSC.

Embodiment 16 discloses embodiments 13-15, wherein the organoids are patient derived and generated from tumor tissue from the patient.

Embodiment 17 discloses embodiments 14-16, wherein the culturing of the organoids on organotypic tissue slice culture is conducted on a semipermeable membrane.

Embodiment 18 discloses embodiments 14-17, wherein the organotypic tissue slice culture is cultured alone for 1-2 weeks prior to culturing with the organoids.

Embodiment 19 discloses embodiments 13-18, wherein the organoid is between 500 and 600 μm.

Embodiment 20 discloses embodiments 13-19, wherein determining the efficacy of the alternating electric field comprises determining the expression of a marker associated with cell proliferation, wherein a decrease in expression of the marker in the organoid indicates the alternating electric field is effective.

Embodiment 21 discloses embodiments 13-19, wherein determining the efficacy of the alternating electric field comprises measuring a size of the organoid prior to and after applying the alternating electrical field, wherein a decrease in the size of the organoid after applying the alternating electrical field indicates the alternating electric field is effective.

Embodiment 22 discloses embodiments 13-21, further comprising applying alternating electric fields to a target site of a subject for a period of time, the alternating electric fields having a frequency and field strength, wherein the organoids are grown from tumor tissue derived from the subject, wherein the target site comprises cancer cells.

Embodiment 23 discloses a method of determining the efficacy of alternating electric fields on a subject comprising culturing a tumor slice from the subject, applying alternating electric fields to the tumor slice for a period of time, the alternating electric fields having a frequency and field strength, and determining the efficacy of alternating electric fields.

Embodiment 24 discloses embodiment 23, wherein culturing the tumor slice occurs on a semipermeable membrane.

Embodiment 25 discloses embodiments 23-24, wherein determining the efficacy of the alternating electric field comprises determining the expression of a marker associated with cell proliferation, wherein a decrease in expression of the marker in the tumor slice indicates the alternating electric field is effective.

Embodiment 26 discloses any of the previous embodiments, wherein the frequency of the alternating electric field is between 50 kHz and 1 MHZ.

Embodiment 27 discloses any of the previous embodiments, wherein the frequency of the alternating electric field is 150 or 250 kHz.

Embodiment 28 discloses any of the previous embodiments, wherein the alternating electric field has a field strength of between 0.5 and 4 V/cm RMS.

Embodiment 29 discloses a method of determining or selecting a value of a parameter of an alternating electric field to be administered for treating a disease or condition, the method comprising: applying an alternating electric field to at least two microtumors produced from cells obtained from a subject, wherein the alternating electric field applied to each of the at least two microtumors has a different frequency from the other, and determining which frequency is most effective at reducing viability of cancer cells in the microtumors.

Embodiment 30 discloses a method of determining or selecting a value of a parameter of an alternating electric field to be administered for treating a disease or condition, the method comprising: applying an alternating electric field to at least two organoids produced from cells obtained from a subject, wherein the alternating electric field applied to each of the at least two organoids has a different frequency from the other, and determining which frequency is most effective at reducing viability of cancer cells in the organoids.

Embodiment 31 discloses a method of determining or selecting a value of a parameter of an alternating electric field to be administered for treating a disease or condition, the method comprising: applying an alternating electric field to at least two tumor slices produced from cells obtained from a subject, wherein the alternating electric field applied to each of the at least two tumor slices has a different frequency from the other, and determining which frequency is most effective at reducing viability of cancer cells in the tumor slices.

Embodiment 32 discloses embodiments 29-31, wherein the parameter of the alternating electric field is selected from application time, frequency, and field strength.

Embodiment 33 discloses embodiments 29-32, wherein the period of time is 72 to 96 hours.

Embodiment 34 discloses embodiments 29-33, wherein the frequency of the alternating electric fields is between 50 kHz and 1 MHZ.

Embodiment 35 discloses embodiments 29-34, wherein the field strength is between 0.5V/cm and 2V/cm.

Embodiment 36 discloses a method of treating a subject having cancer comprising determining the efficacy of alternating electric fields in an ex vivo model derived from the subject, and applying alternating electric fields to the subject when the alternating electric fields are determined to be effective in the ex vivo model

Embodiment 37 discloses embodiment 36, wherein determining the efficacy of alternating electric fields is performed using the methods of one or more of claims 1-28.

Embodiment 38 discloses a method of treating a subject having cancer comprising determining the parameters of an alternating electric field effective for treating the cancer in the subject, and applying the alternating electric fields, using the parameters determined to be effective, to a target site of the subject, wherein the target site comprises one or more cancer cells.

Embodiment 39 discloses embodiment 38, wherein determining the parameters of alternating electric fields is performed using the methods of one or more of claims 29-35.

Embodiment 40 discloses a method of identifying a biomarker that responds to application of an alternating electric field comprising: performing a morphological or molecular analysis of a microtumor formed from primary cancer cells from a subject; applying an alternating electric field to the microtumor for a period of time, the alternating electric field having a frequency and field strength; performing a morphological or molecular analysis of the microtumor after step c); and comparing the morphological or molecular analysis of step b) with the morphological or molecular analysis of step d), wherein a difference in the morphological or molecular analysis of step b) with the morphological or molecular analysis of step d) identifies a biomarker that responds to application of an alternating electric field.

Embodiment 41 discloses a method of identifying a biomarker that responds to application of an alternating electric field comprising: performing a morphological or molecular analysis of an organoid formed from cancer tissue from a subject; applying an alternating electric field to the organoid for a period of time, the alternating electric field having a frequency and field strength; performing a morphological or molecular analysis of the organoid after step c); and comparing the morphological or molecular analysis of step b) with the morphological or molecular analysis of step d), wherein a difference in the morphological or molecular analysis of step b) with the morphological or molecular analysis of step d) identifies a biomarker that responds to application of an alternating electric field.

Embodiment 42 discloses method of identifying a biomarker that responds to application of an alternating electric field comprising: performing a morphological or molecular analysis of a tumor slice obtained from a subject; applying an alternating electric field to the tumor slice for a period of time, the alternating electric field having a frequency and field strength; performing a morphological or molecular analysis of the tumor slice after step c); and comparing the morphological or molecular analysis of step b) with the morphological or molecular analysis of step d), wherein a difference in the morphological or molecular analysis of step b) with the morphological or molecular analysis of step d) identifies a biomarker that responds to application of an alternating electric field.

### EXAMPLES

### A. Example 1: Glioblastoma-Derived 3-Dimensional Ex Vivo Models to Evaluate Effects and Efficacy of Tumor Treating Fields (TTFields)

### 1. Introduction

Glioblastoma (GBM) is one of the most aggressive primary brain tumors in adults with a median survival time of 16-18 months and a five-year survival rate of 6% for male and 9% for female patients (Wick et al., 2021). During their limited lifespan, patients suffer from neurological deficits like hemiparesis, aphasia, seizures and changes of personality, rendering the disease even more devastating (Fritz et al., 2016; Wick et al., 2021). Maximum standard therapy includes extensive surgery, if functionally possible, followed by radiotherapy combined with concomitant and adjuvant chemotherapy with temozolomide (TMZ) (Stupp et al., 2005; Stupp et al., 2009). However, despite vigorous efforts in research during the last years, relapse is unavoidable and prognosis is even worse in patients with a multifocal involvement (Patil et al., 2012; Stupp et al., 2005) or an unmethylated 06-methylguanine-DNA methyltransferase (MGMT) promoter (Esteller et al., 2000; Rivera et al., 2010). MGMT is a repair enzyme counteracting TMZ chemotherapy (Feldheim et al., 2019).

Tumor Treating Fields (TTFields) therapy, which is applied via arrays placed onto the patient's shaved scalp and utilize alternating electric fields with a frequency of 100-400 kHz and an intensity of 1-3 V/cm, is a new physical treatment modality for a diverse range of cancers (Carrieri et al., 2020; Rominiyi et al., 2020). Interference with the spindle apparatus and disturbance of cytokinesis and thereby hindrance of mitosis and reduced cell proliferation have been suggested as their primary mode of action (Kirson et al., 2004; Kirson et al., 2007; Riley et al., 2019). In addition, several other effects caused by TTFields on the cellular level have been described (Kissling and Di Santo, 2020; Rominiyi et al., 2020). The randomized multicenter EF-14 phase III trial proved the efficacy of TTFields at 200 kHz for the treatment of newly diagnosed GBM when applied with maintenance TMZ chemotherapy following maximal safe surgical resection and chemo-radiation (Stupp et al., 2017). The Kaplan-Meier curves from this trial imply that there are patients who respond very well to TTFields and other less responsive patients. However, it is still unknown, which patients benefit the most from TTFields. Therefore, it might be crucial to identify patients who are likely to benefit from TTFields treatment prior to TTFields application. Since acquisition of matched treatment-naïve and recurrent patient tissues is a challenge, molecular effects of TTFields have been mainly investigated in cell cultures or animal models (Kissling and Di Santo, 2020) and information on mechanisms leading to TTFields resistance is scarce. Thus, investigating how TTFields alter the tumor biology in humans is a so far insufficiently met necessity, and reliable organotypic ex vivo test systems, which will allow for a controlled and repeatable evaluation of the effects and efficacy of TTFields in the laboratory are critical.

Consequently, described herein are three different patient derived 3D ex vivo models for TTFields studies. Patient-derived material can be cultured and used as a representative surrogate in order to investigate TTFields function ex vivo: (1) GBM-patient derived primary cells (PDPC) can be seeded onto murine organotypic hippocampal slice cultures (OHSC) to form microtumors (Schulz et al., 2021). (2) Organoids derived from intraoperatively resected material can be cultured up to several months, frozen and thawed while maintaining the tumor-inherent invasive, immunohistological, cellular and mutational profile (Jacob et al., 2020). For TTFields application, they are placed onto OHSC as a tissue carrier and grown as microtumors. (3) Organotypic tumor slice cultures generated from fresh intraoperative material that represents patient-specific tumor properties and the tumor microenvironment for a more realistic setup, can be preserved up to 6 days (Merz et al., 2013).

The aim of the present study was to investigate whether the implementation of PDPC or patient derived organoids, as well as organotypic tumor slice cultures is technically feasible as respective 3D-model systems for testing the effect and efficacy of TTFields. This approach can be used as a test system prior to TTFields treatment in order to determine patients' response to therapy, optimize treatment such as testing possible combinations or optimal frequencies, and to investigate molecular mechanisms of treatment response and resistance.

### 2. Materials and Methods

i. Tissue Samples

All patients were newly diagnosed, did not receive any prior tumor treatment and were operated at the Department of Neurosurgery, University Hospital Würzburg, Germany. They gave written informed consent in accordance with the declaration of Helsinki, and as approved by the Institutional Review Board of the University of Würzburg (#22/20-me). The histopathology of the tumor samples was confirmed by an experienced neuropathologist and classified according to the 2021 WHO criteria (Louis et al., 2021). Only GBM, IDH-wildtype CNS WHO grade 4 samples were included.

ii. Patient Derived Primary Cells, Cell Lines, and Cell Culture

To generate PDPC, necrotic areas and blood vessels were removed from the intraoperatively obtained tumor tissue and the latter then separated using a homogenizer. The homogenized tumor material was cultured in 25 cm3 cell culture flasks (Corning, New York, NY, USA) in Dulbecco's Modified Eagle's Medium (DMEM) containing 1 g/l glucose, sodium pyruvate, 3.7 g/l NaHCO3 and L-glutamine and supplemented with 20% v/v heat-inactivated fetal calf serum (FCS), 2× non-essential amino acids (NEAA, 100× stock, add 10 ml to 500 ml medium) (all from Gibco, Carlsbad, USA) and 1.5% vitamin C (Sigma-Aldrich, St. Louis, USA) at 37° C., 5% CO2 and 95% humidity until an adherent cell layer was formed (Hagemann et al., 2017). GBM cell line U87MG (CLS, Eppelheim, Germany) was cultured in DMEM supplemented with 10% v/v FCS, 2× NEAA, 3 mM L-glutamine, 100 U/ml penicillin, and 100 mg/ml streptomycin (Invitrogen, Carlsbad, USA) as a monolayer under the same conditions.

iii. Generation of Organoids

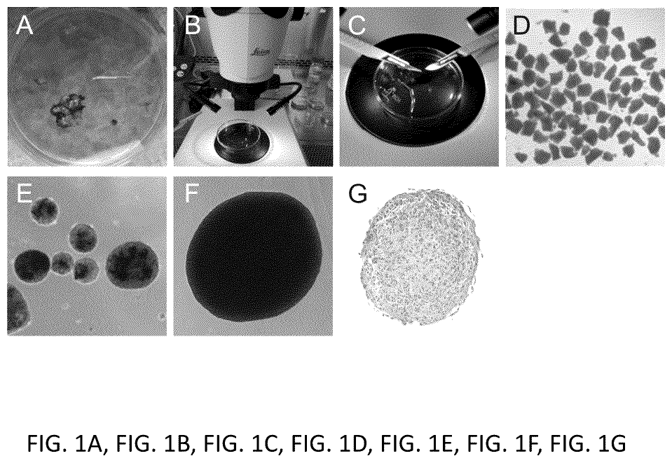

Organoids were prepared according to the method described by Jacob et al. (Jacob et al., 2020). Fresh intraoperatively obtained tumor tissue was temporarily stored on ice in Hibernate A medium (Gibco, Carlsbad, USA) (FIG. 1A). Next, the tissue was cleared of necrosis and blood vessels and carefully minced with a scalpel into approximately 0.5 mm large pieces under the microscope (FIG. 1B-D). These pieces were then treated with RBC Lysis Buffer (Invitrogen, Carlsbad, USA) for 10 min and washed two times with Hibernate A medium containing 1% Glutamax. 0.4% penicillin/streptomycin and 0.1% Amphotericin (HGPSA) (all from Gibco, Carlsbad, USA). Sections were transferred into GBO medium consisting of 47.24% DMEM/F12, 47.25% Neurobasal, 0.02% B27 without Vitamin A (50x), 0.01% Glutamax. 0.01% N2, 0.01% NEAA, 0.004% penicillin/streptomycin, 0.001% B-Mercaptoethanol (all from Gibco, Carlsbad, USA) and 0.00023% human insulin (Sigma-Aldrich, St. Louis, USA) to ultra-low attachment 6-well plates (Corning Costar, New York, USA) and incubated at 37° C., 5% CO2 and 95% humidity on an orbital shaker at 120 rpm. After 2 weeks of culture, organoids formed successfully and could be used for further experiments (FIG. 1E-G).

iv. Preparation of Organotypic Hippocampal Brain Slice Cultures (OHSC)

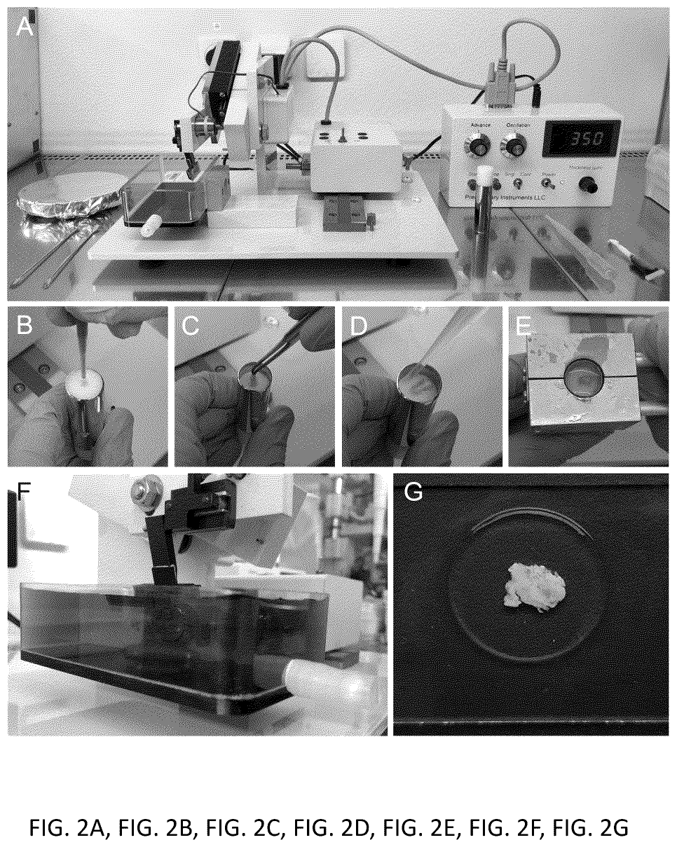

OHSC were prepared as described previously (Schulz et al., 2021). Briefly, mice 5-8 days postpartum (p5-p8) were decapitated following ethical guidelines. The brain was dissected under the microscope and glued with the dorsal surface facing downward into the sample tube of the vibratome (Precisionary Instruments, Greenville, USA) (FIG. 2A) with histoacryl glue (B. Braun, Tuttlingen, Germany) (FIG. 2B, C). The tube was filled with molten agarose (Sigma-Aldrich, St. Louis, USA) (FIG. 2D). A cooling block pre-cooled at −80° C. and then stored on ice till usage ensured rapid hardening of the agarose (FIG. 2E). The sample tube was clamped into the vibratome, which was set to an advance of 3.5 and an oscillation of 6 and 350 μm thick slices were generated (FIG. 2F). Slices were collected in the preparation tray filled with minimal essential medium (MEM) supplemented with 1% penicillin/streptomycin, 1% L-glutamine (all from Gibco, Carlsbad, USA) and 1% glucose (Sigma-Aldrich, St. Louis, USA). The hippocampus was isolated, transferred using a wide glass pipette and cultured on inserts with a semi-permeable membrane of 0.4 μm pore size (Greiner Bio-one, Frickenhausen, Germany) in a 24-well plate (Corning Costar, New York, USA) containing brain slice medium (MEM supplemented with 25% normal horse serum, 25% Hank's Balances salt solution (HBSS), 1% penicillin/streptomycin, 1% L-glutamine (all from Gibco, Carlsbad, USA), vitamin C and 1% glucose (both from Sigma-Aldrich, St. Louis, USA)) at 35° C., 5% CO2 and 95% humidity. It was vital that excess medium had to be aspirated from the OHSC. The slices obtain their nutrient supply from the medium via the pores of the insert and must not be covered by any liquid. In addition, the slice should be placed as centrally and planar as possible onto the insert. After a cultivation period of two weeks, experiments could be started.

v. Seeding of Fluorescence-Labeled GBM Cells and Organoids onto OHSC

The cells were fluorescently labeled for easy visualization of U87MG cells, PDPC and organoids growing on OHSCs. U87MG and PDPC were transfected with green fluorescent protein (GFP) utilizing the pmaxGFP plasmid (Lonza, Cologne, Germany) in combination with nucleofection using the Amaxa Cell Line Nucleofector Kit V (Lonza, Cologne, Germany) as detailed elsewhere (Hagemann et al, 2006; Hagemann et al. 2017). Briefly, cells were detached with 0.25% trypsin/EDTA (Carl Roth, Karlsruhe, Germany) and suspended in cell culture medium. For each transfection, 1×10⁶ cells were centrifuged for 10 min at 300× g at room temperature. The supernatant was discarded, and the cells were re-suspended in 100 μl of Nucleofector solution V. In a 1.5 ml reaction tube, cells were mixed with 2 μg pmaxGFP plasmid, transferred to transfection cuvettes and electroporated with transfection program U29. After transfection, cells were transferred to the wells of a 6-well plate containing 1 ml cell culture medium each. The cells were allowed to recover for 2-3 days before further use. Approximately 1×10⁵ cells were taken up in 10 μl cell culture medium and spread onto the surface of the OHSC. After 2-3 days, microtumor growth and invasion could be detected using the inverted fluorescence microscope LEICA DMI 3000 B (Leica, Wetzlar, Germany).

Organoids were mechanically minced with scalpels into pieces of 100 μm size and incubated in 10 μM Carboxyfluorescein succinimidyl ester (CFSE) in PBS for 15 min as provided in the CellTrace™ CFSE Cell Proliferation Kit (Invitrogen, Carslbad, USA) and following the manufacturer's instructions. The solution was then replaced with fresh GBO medium and incubated at 37° C. for another 30 min. The next day, organoids fluoresced at an excitation wavelength of 488 nm and were placed onto the OHSC using a pipette.

vi. Generation of Patient Derived Organotypic Tumor Slice Cultures

Generation of organotypic tumor slice cultures is based on a publication by Merz et al. (Merz et al., 2013). After surgical tumor resection, the tissue was directly transferred to Hibernate A medium and stored on ice. The tumor tissue was carefully freed from necrosis and blood vessels and cut into approximately 2 cm×0.5 cm pieces using a scalpel. Preparation of slices was performed as described above for the OHSC (FIG. 2). The slices were embedded in agarose (FIG. 2G) and had to be carefully cut out with a scalpel before they could be transferred to the center of the semipermeable membrane with a wide glass pipette. Vitality of slices, histopathology and tumor content was assessed by an experienced neuropathologist. Tumor slices could be cultured in brain slice medium at 35° C., 5% CO₂ and 95% humidity for up to 6 days. The medium was changed every second day.

vii. TTFields Treatment

The Inovitro™ laboratory research system (Novocure, Haifa, Israel) was used for TTFields administration. U87MG cells and PDPC cultured as monolayers were treated as described previously (Kessler et al., 2018). Briefly, glass coverslips with 20 mm diameter (Hartenstein, Würzburg, Germany) were placed into linovitroTMceramic dishes (Novocure, Haifa, Israel). Cells were trypsinized and plated onto the coverslips by placing 350 μl cell culture medium containing 30,000 cells as a drop in their center. Cells attached during 20 h incubation at 37° C. and 5% CO₂. The medium was replaced by 2 ml fresh cell culture medium, the ceramic dishes were placed onto a base plate connected to a TTFields generator and TTFields at 200 kHz were applied with an intensity of 1.7 V/cm for 72 h. The medium was renewed every 48 h. Control cells were kept under the same conditions without TTFields application. To evaluate TTFields effects, cells were trypsinized and counted using the Scepter 2.1 cell counter (Merck, Darmstadt, Germany).

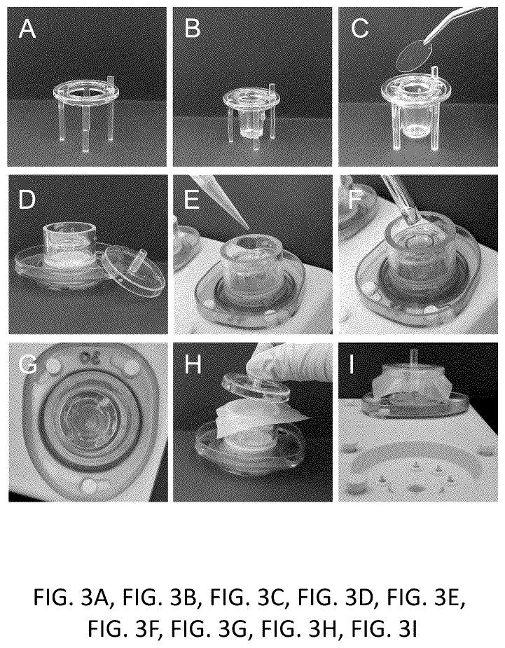

To treat OHSC and tumor slices with TTFields, ceramic dishes with high walls (Novocure, Haifa, Israel) were utilized (FIG. 3). The holders containing the inserts with semipermeable membranes were placed into the dishes and 2.5 ml brain slice medium was pipetted into the dishes outside the inserts (FIG. 3A-E). OHSC and tumor slices, respectively, were transferred as described above (FIG. 3F, G). To avoid condensation water to drop onto the slice surfaces, a 12 mm coverslip was placed onto each insert. The ceramic dish was covered with parafilm and closed with a lid, to minimize evaporation of the medium (FIG. 3C, H, I). TTFields were applied for 72 to 96 h at 200 kHz and 1.5 V/cm. Condensation water was carefully aspirated every day, the medium was changed every second day. To evaluate microtumor growth on OHSC, images were taken using the LEICA DMI 3000 B microscope, LEICA DFC450 camera and LAS V4.5 software (all Leica, Wetzlar, Germany). Tumor size was determined on the fluorescence images using the Measure Tool from the open source program Fiji (Image J 1.53c) (Schindelin et al., 2012; Schneider et al., 2012). Tumor slices were fixed in 4% formalin (Carl Roth, Karlsruhe, Germany) for 24 h at 4° C. and then transferred to phosphate buffered saline (PBS) (Sigma-Aldrich, St. Louis, USA) for immunohistochemical and immunofluorescence staining.

viii. Immunohistochemical and Immunofluorescence Staining

The fixed brain slices were dehydrated, embedded in paraffin, and sliced into 3 μm thick sections. Next, standardized hematoxylin and cosin (HE) (Carl Roth, Karlsruhe, Germany) staining was performed for histology. For immunohistochemical staining. Ki67 (ab16667, Abcam, Cambridge, UK) and GFAP (sc33673, santacruz, Dallas, USA) antibodies were used at a dilution of 1:1,000 and 1:100 in antibody dilution buffer (DCS Innovative Diagnostik Systeme, Hamburg, Germany), respectively, and incubated overnight at 4° C. Protein expression was visualized using the secondary antibodies AlexaFluor488 and AlexaFluor555 (both from Invitrogen, Carlsbad, USA) in a 1:1,000 dilution and incubated for 1 h at room temperature. Finally, the slices were mounted using Fluoroshield mounting medium containing DAPI (Abcam, Cambridge, UK). Five representative fields of view per slide were photographed with the LEICA DMI 3000 B microscope with standardized settings at an 40× amplification and analyzed for staining intensity via the batch processing function of the open source program Fiji (ImageJ 1.53c) (Schindelin et al., 2012; Schneider et al., 2012). The macro settings are described elsewhere (Feldheim et al., 2018).

ix. Statistical Analysis

Statistical analysis was performed using GraphPadPrism 9 software (GraphPad Software, San Diego, USA). Statistical significance was defined by unpaired 2-tailed t-tests, and by ANOVA. P<0.05 was considered to be significant. In the box plots the boxes represent the median with the 25% and 75% quartile and the whiskers the minimum and maximum of the data set. Descriptive statistics was performed to calculate the tumor size and its growth or decrease, the mean value was expressed as percentage with standard error of the mean (±SEM). The difference was also shown with ±SEM as well as the 95% confidence interval (CI). All experiments have been performed at least in triplicates.

### 3. Results

i. Patient Cohort

To establish the described ex vivo GBM models, tumor samples of eight GBM patients were utilized (Table 1). The tumors were classified according to the most recent WHO classification of tumors of the central nervous system (Louis et al., 2021). Only GBM, IDH-wildtype CNS WHO grade 4 were used. A low amount of tumor material prevented execution of all models with the same samples in this proof of principle study.

TABLE 1
Clinical parameters of glioblastoma samples and the models they were utilized for.

| | | age | | | Ki67 | MGMT promoter | | IDH1 | IDH2 | ATRX | |
|---|---|---|---|---|---|---|---|---|---|---|---|
| ID | sex | [years] | histology | KPI | [%] | methylation [%] | | mutation | mutation | expression | experiment |
| 1 | male | 64 | GBM | 100 | 20 | yes | (76) | no | no | yes | PDPC |
| 2 | female | 79 | GBM | 100 | 20 | yes | (66) | no | no | yes | PDPC |
| 3 | female | 73 | GBM | 100 | 50 | yes | (28) | no | no | yes | PDPC |
| 4 | female | 69 | GBM | 30 | 25 | yes | (71) | no | no | yes | organoid |
| 5 | female | 65 | GBM | 90 | 20 | no | (8.0) | no | no | yes | organoid |
| 6 | male | 65 | GBM | 90 | 40 | no | (4.0) | no | no | yes | organoid |
| 7 | male | 64 | GBM | 40 | 30 | yes | (82) | no | no | yes | tumor slice |
| 8 | male | 54 | GBM | 90 | 40 | no | (3) | no | no | yes | tumor slice |

ID=identification number; KPI=Karnofsky performance index; MGMT=O6-methylguanine-DNA methyltransferase; IDH=isocitrate dehydrogenase; ATRX=a thalassemia/mental retardation syndrome X-linked; GBM=glioblastoma; PDPC=patient derived primary cells.
ii. Patient Derived GBM Primary Cells Display Variable Responses to TTFields at 200 kHz

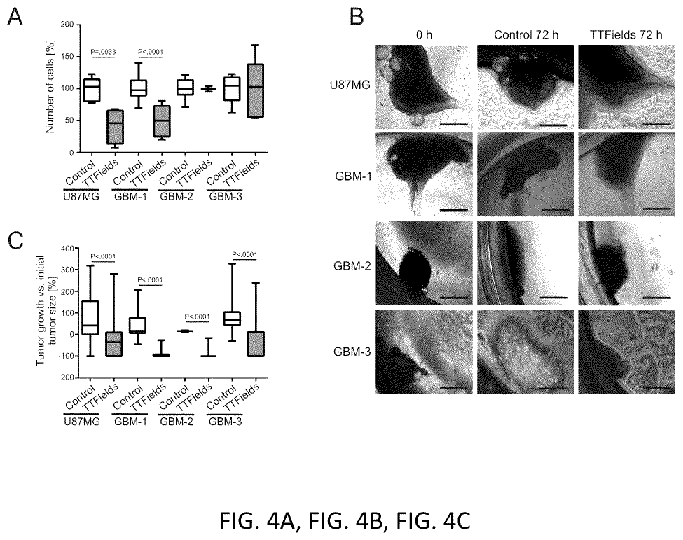

Previously published data revealed that application of TTFields at 200 kHz significantly decreased the number of GBM cells (Kirson et al., 2004; Giladi et al., 2015; Kirson et al., 2007; Kessler et al., 2018). In these experiments, U87MG cells and three PDPC monolayer cell cultures were treated with TTFields at 200 kHz for 72 h. These cells responded to TTFields to a variable extent (FIG. 4A). Whereas the cell numbers of U87MG cells were reduced to 41.75%±13.76% compared to the untreated control (P=0.0033, CI=−90.76 to −25.74) and those of GBM-1 were diminished to 49.7%±10.87% (P<0.0001, CI=−69.19 to −31.41), GBM-2 and GBM-3 were non-responsive to TTFields treatment (FIG. 4A).

These cells were then seeded onto OHSC to investigate whether a 3-dimensional growth in form of microtumors will reproduce these results. Three days after seeding, microtumors had formed (FIG. 4B) and were subjected to TTFields at 200 kHz for 72 h. Indeed, the response of U87MG and GBM-1 microtumors, respectively, was confirmed (FIG. 4B, C). During the 72 h observation period, U87MG microtumors in the no treatment control grew on average by 72.84%±18.01%. In contrast, TTFields led to shrinkage of microtumors by −28.59%±7.72%, which is a difference between the averages of −101.4±16.72 percentage points (P<0.0001, CI=−134.6 to −68.29) (FIG. 4C). Similarly, GBM-1 microtumors grew by 36.5%±19.07% if not treated with TTFields, whilst the treated microtumors downsized by −90.37%±4.82%, a difference of −126.9±16.19 percentage points (P<0.0001, CI=−160.0 to −93.75) (FIG. 4C). Surprisingly, GBM-2 and GBM-3 microtumors were responsive to TTFields, when grown in form of microtumors. GBM-2 microtumors grew by 15.5%±3.5% when they remained untreated, but shrank −90.72%±6.37% when subjected to TTFields, a difference of −106.2±19.58 percentage points (P<0.0001, CI=−147.4 to −65.09). The response of GBM-3 was very similar. Control microtumors grew by 100.6%±27.57%, while TTFields treated tumors reduced in size by −37.64%±14.64%, which is a difference of −138.2±28.78 percentage points (P<0.0001, CI=−196.1 to −80.4) (FIG. 4C).

iii. TTFields Induce Shrinkage of Patient Derived GBM-Organoids Growing on OHSC

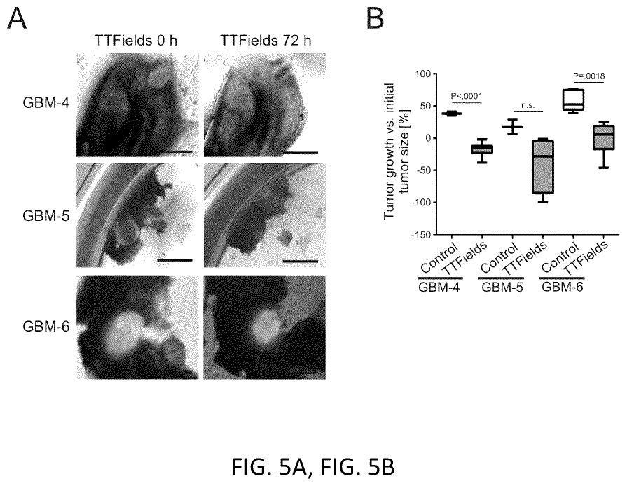

Above data indicate that GBM cells growing as 3-dimensional microtumors exhibit an increased sensitivity towards TTFields. Therefore, to be even closer to the tumor tissue-like 3-dimensional structure, organoids generated from fresh, intraoperatively gained GBM tissue of three different patients were grown on OHSC as carrier tissue. The organoids of all patients grew into the OHSC within 2-3 days but displayed a heterogeneous appearance and growth pattern after TTFields treatment at 200 kHz for 72 h (FIG. 5A). GBM-4 organoids decreased in size by −17.10%±3.07%, whereas the untreated control organoids grew by 38.0%±3.0%. This was a difference in the average organoid size of −55.10±7.21 percentage points (P<0.0001, CI=−71.17 to −39.03). Although the organoids of GBM-5 were clearly shrinking by −39.68%±3.0% under TTFields treatment, the difference of −57.76±33.41 percentage points to the control organoids, which grew by 18.08%±11.30%, was not statistically significant (P=0.1589, CI=−150.5 to 35.01. In contrast, TTFields treated GBM-6 organoids did not shrink, but were stalled in their growth (−0.003%±10.55%), whereas control organoids grew by 58.12%±7.13%. This was a statistically significant difference of −58.12±13.32 percentage points (P=0.0018, CI=−88.26 to −27.98) (FIG. 5B).

iv. Cell Proliferation Decreases and Apoptosis Increases in Human Organotypic GBM Tumor Slice Cultures when Treated with TTFields

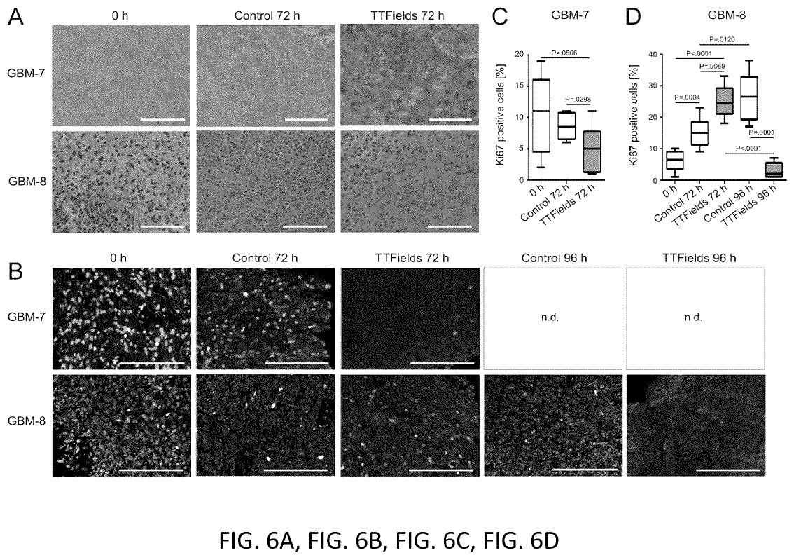

Human organotypic GBM tumor slice cultures were treated with TTFields at 200 kHz for 72 h. HE staining revealed a general decrease in cell number and a concomitant increase of apoptotic cells in the treated slices compared to untreated controls (FIG. 6A). While staining for the proliferation marker Ki67 remained basically constant over time in GBM-7 (mean expression 10.38%±2.14% at 0 h vs. 8.63%±0.71% 72 h later) and only slightly dropped to 5.0%±1.32% (P=0.0298, CI-6.84 to −0.41) when TTFields were applied (FIG. 6B, C), GBM-8 displayed a completely different picture. There was an increase of Ki67 positivity by 9.17±1.994 percentage points from 6%±1.99% (0 h) to 15.17%±2.01% (72 h) observable not only in untreated control slices (P=0.0004, CI 4.89 to 13.44), but even more pronounced by 19.0±2.05 percentage points to 25.0%±2.1% after 72 h TTFields treatment (P <0.0001, CI 14.61 to 23.39) (FIG. 6B, D). Ki67 staining only identifies cell cycle entry without telling, whether the cell cycle will be completed (Thomasova and Anders, 2015; Li et al., 2015). Since TTFields have been shown to induce a G1 cell cycle arrest (Voloshin et al., 2016), this increase in Ki67 was examined to see if it will sustain and incubated the GBM-8 slices for another 24 h with TTFields. At 96 h total treatment, the percentage of Ki67 positive cells within the slice dropped to 3.0%±1.14% (P<0.0001, CI-27.74 to −16.26), while Ki67 raised to 26.5%±3.12% in control cells (P=0.0120, CI 3.09 to 19.57) (FIG. 6B, D).

### 4. Discussion

TTFields added to the standard maintenance therapy improve the median progression-free and overall survival of newly diagnosed GBM patients by 2.7 and 4.9 months, respectively. Above that, they more than doubled the 5-year survival from 5% to 13% (Stupp et al., 2017). TTFields are mostly well tolerated with systemic toxicity for TTFields plus standard treatment group being comparable to the standard therapy group and mild to moderate skin toxicity, e.g. skin rash and eczema underneath the arrays, occurring in 52% of patients in the TTFields group (Lacouture et al., 2014; Stupp et al., 2017). In order to reach highest efficacy, TTFields should be applied for ≥18 h each day on average (Toms et al., 2019). It has been discussed that carrying the device of 1.3 kg on a daily basis might restrict patients from daily activities and might hamper social life (Kwan et al., 2018). In addition, to place the arrays patients need to shave their scalps, which might lead to stigmatization (Kwan et al., 2018). However, preliminary data of the recent TIGER trial do not support such apprehensions (Bähr et al., 2022). Nevertheless, not only the TTFields hardware itself is costly, but also maintenance is expensive (Rominiyi et al., 2020). Not all patients respond to TTFields to the same extent, some gain only a slight advantage, while others survive for 5 years or longer (Stupp et al., 2017). A standard subgroup analysis based on e.g. the MGMT promoter methylation status, extent of resection, Karnofsky performance index (KPI) or age failed to identify which patients respond better to TTFields, as a survival benefit was demonstrated in all subgroups (Stupp et al., 2017). Nevertheless, patients who benefit from TTFields should be chosen wisely in order to allocate TTFields treatment efficiently. Since information on mechanisms leading to TTFields resistance is very limited and most molecular effects of TTFields have been investigated in cell cultures or animal models only (Kissling and Di Santo, 2020), the analysis of patients' tumor samples before and after TTFields treatment would be desirable. However, it is known that there might be a bias amongst such patients. Their TTFields device usage might vary (Toms at al., 2019) as well as the total treatment duration (Stupp et al., 2017). In addition, the time between end of TTFields treatment and operation of the relapse differs from patient to patient. Additionally, patients with a reduced KPI might not be re-operated, while the time till relapse and following re-surgery might be prolonged in other patients. These factors might limit the availability of tissue for research. On the other hand, tumor samples of especially these patient groups are most interesting in terms of investigating treatment resistance, related molecular and cellular differences of interpatient tumor heterogeneity and post-therapeutic changes.

To address these limitations, several patient derived ex vivo tumor tissue culture methods can be used in order to identify patients who might benefit from TTFields therapy prior treatment and to investigate short- and long-term effects of TTFields including molecular prerequisites leading to TTFields responsiveness or resistance. At first, the standard GBM cell line U87MG and different PDPC monolayer cultures were treated with TTFields. As expected, there was a high variability of TTFields response with some PDPC not reacting at all, putatively reflecting the patients' tumor sensitivity for TTFields treatment. However, when grown as microtumors on OHSC, the cells became more sensitive. Thus, 3-dimensional growth might better represent the in vivo situation and lead to more reliable results. Nevertheless, while this primary cell only approach might be easily done from a technical point of view, it has restricted validity as a screening system for clinical application. Due to the rupture of cell-cell-contacts during lysis, duration of cultivation, lack of tumor microenvironment and hypoxic gradients, PDPC cultures not only change their antigen surface expression patterns, but also undergo molecular and transcriptional changes and thus no longer represent the parental tumor characteristics (Jacob et al., 2020). New ex vivo models like organoids or tumor slice cultures can be used.

Organoids represent the histological characteristics, cellular diversity, gene expression, and mutational profiles of their corresponding parental tumors. In addition, they can be generated quickly and reliably within two weeks from intraoperatively gained tissue (Jacob et al., 2020). Thus, they would be available for TTFields testing already two weeks after surgery and results of TTFields screenings can be expected at a time when patients completed radiotherapy and could start with TTFields application. The data show that organoids grown on OHSC respond to TTFields treatment by interpatient heterogeneous appearance and growth patterns with TTFields causing shrinkage of the microtumors to varying extent. Thus, patient derived GBM tumor organoids represent an ideal and flexible model to not only test personalized therapies by correlating mutational profiles with responses to TTFields, but also to investigate changes in organoid cell structures and molecular protein expression patterns caused by TTFields. Since organoids can be propagated over long time periods without changing their properties (Jacob et al., 2020) they also should be suitable to investigate long-term TTFields effects in a patient related setting.

In addition to the above mentioned paternal specifics, patient derived organotypic tumor slice cultures retain the tumor microenvironment including neural cells and therefore, are even closer to the in vivo tumor situation of GBM patients (Merz et al., 2013). Freshly sliced, the tissue is ready to use, but on the downside, slicing is a delicate method highly dependent on tissue quality and susceptible to deficiencies. These cultures require a high level of experience and they are viable only for a couple of days. On the other hand, adjacent slices from the same tumor region can be generated and cultured for better comparison of different experimental conditions. Especially molecular alterations can be visualized in slice cultures. The slice cultures were stained for Ki67, a well-established proliferation marker in pathological evaluation of tumor tissue (Li et al., 2015). It was intriguing that the percentage of Ki67 positive cells in one of the investigated GBM samples was increasing over time despite TTFields treatment, which is expected to interfere with cell proliferation (Kirson et al., 2004; Kirson et al., 2007; Riley et al., 2019). However, Ki67 staining identifies cells which entered the cell cycle and is high in G1, S, G2 and M phase, but does not give an indication about the cells' later fate (Thomasova and Anders, 2015; Li et al., 2015). TTFields can cause a cell cycle arrest, which might lead to accumulation of Ki67 positive cells for a certain time. Indeed, when staining the slices after 96 h instead of 72 h treatment, there was a massive drop in Ki67 positivity, probably due to Ki67 degradation and cell death. While this observation is based on only one single tumor and should not be overinterpreted, it proves the feasibility of investigating TTFields-induced molecular changes in cultured tumor tissue slices. Therefore, both organoids and tumor slice cultures have different advantages, which complement each other.

### 5. Conclusions

This study identified differences in the interpatient treatment response not only when using PDPC cultures, but especially when utilizing patient derived organoids growing on OHSC and tumor slice cultures. This establishes the methodology as a powerful tool to screen for patients that might benefit from TTFields treatment as well as to elucidate cellular alterations within the cultured tissue. Additionally, these models can be used to shed light onto molecular mechanisms of treatment response and resistance, especially if they are used combined.

Those skilled in the art will recognize, or be able to ascertain using no more than routine experimentation, many equivalents to the specific embodiments of the method and compositions described herein. Such equivalents are intended to be encompassed by the following claims.

### REFERENCES

Bähr O; Tabatabai G. Fietkau R. Goldbrunner R. Glas M. First results of the TIGER study—treatment decision and quality of life of glioblastoma patients during TTFields therapy in routine clinical care. Deutsche Gesellschaft für Neurochirurgie. 73. Jahrestagung der Deutschen Gesellschaft für Neurochirurgie (DGNC), Joint Meeting mit der Griechischen Gesellschaft für Neurochirurgie. Köln, 29.05.-01.06.2022. Düsseldorf: German Medical Science GMS Publishing House 2022. DocV228. doi: 10.3205/22dgnc220

Carieri F A, Smack C, Siddiqui I, Kleinberg L R, Tran P T. Tumor treating fields: at the crossroad between physics and biology for cancer treatment. Front Oncol 2020. 10:575992. doi: 10.3389/fonc.2020.575992

Esteller M, Garcia-Foncillas J, Andion E, Goodman S N, Hidalgo O F, Vanaclocha V, Baylin S B, Herman J G. Inactivation of the DNA-repair gene MGMT and the clinical response of gliomas to alkylating agents. N Engl J Med 2000. 343 (19): 1350-1354. doi: 10.1056/NEJM200011093431901

Feldheim J. Kessler A F, Schmitt D, Wilczek L, Linsenmann T, Dahlmann M, Monoranu C M, Ernestus R-I, Hagemann C, Löhr M. Expression of activating transcription factor 5 (ATF5) is increased in astrocytomas of different WHO grades and correlates with survival of glioblastoma patients. Onco Targets Ther 2018. 11:8673-8684. doi: 10.2147/OTT.S176549. eCollection 2018

Feldheim J, Kessler, A F, Monoranu C M, Ernestus R-I, Löhr M, Hagemann C. Changes of O⁶-methylguanine DNA methyltransferase (MGMT) promotor methylation in glioblastoma relapse-a meta-analysis type literature review. Cancers 2019. 11:1837. doi: 10.3390/cancers11121837

Fritz L, Dirven L, Reijneveld J C, Koekkoek J A F, Stiggelbout A M, Pasman H R W, Taphoorn M J B. Advance care planning in glioblastoma patients. Cancers (Basel) 2016. 8 (11): 102. doi: 10.3390/cancers8110102

Giladi M. Schneiderman R S, Voloshin T, Porat Y, Munster M, Blat R, Sherbo S, Bomzon Z, Urman N, Itzhaki A, Cahal S, Shteingauz A, Chaudhry A, Kirson E D, Weinberg U. Palti Y. Mitotic spindle disruption by alternating electric fields leads to inproper chromosome segregation and mitotic catastrophe in cancer cells. Sci Rep 2015. 5:18046. doi: 10.1038/srep18046

Hagemann C, Amend D, Kessler A F, Linsenmann T, Ernestus R-I, Löhr M. High-efficiency transfection of glioblastoma cells and a simple spheroid migration assay. Methods Mol Biol 2017. 1622:63-79. doi: 10.1007/978-1-4939-7108-4_5

Hagemann C, Meyer C, Stojic J, Eicker S, Gerngras S, Kühnel S, Roosen K, Vince G H. High efficiency transfection of glioma cell lines and primary cells for overexpression and RNAi experiments. J Neurosci Methods 2006. 156 (1-2): 194-202. doi: 10.1016/j.jneumeth.2006.03.003

Herrlinger U, Tzaridis T, Mack F, Steinbach J P, Schlegel U, Sabel M, Hau P, Kortmann R-D, Krex D, Grauer O, Goldbrunner R, Schnell O, Bähr O, Uhl M, Seidel C, tabatabai G, Kowalski T, Ringel F, Schmid-Graf F, Suchorska B, Brehmer S, Weyerbrock A, Renovanz M, Bullinger L, Galldiks N, Vajkoczy P, Misch M, Vatter H, Stuplich M, Schäfer N, Kebir S, Weller J, Schaub C, Stummer, W, Tonn J-C, Simon M, Keil V C, Nelles M, Urbach H, Coenen M, Wick W, Weller M, Fimmers R, Schmid M, Hattingen E, Pietsch T, Coch C, Glas M, Neurooncology Working Group of. Lomustine-temozolomide combination therapy versus standard temozolomide therapy in patients with newly diagnosed glioblastoma with methylated MGMT promoter (CeTeG/NOA-09): a randomised, open-label, phase 3 trial. Lancet 2019. 393 (10172): 678-688. doi: 10.1016/S0140-6736 (18) 31791-4

Jacob F. Salinas R D, Zhang D Y, Nguyen P T T, Schnoll J G. Wong S Z H, Thokala R, Sheikh S, Saxena D, Prokop S, Liu D-A, Qian X, Petrov D, Lucas T, Chen H I, Dorsey J F, Christian K M, Binder Z A, Nasrallah M, Brem S, O'Rourke D M, Ming G-L. Song H. A patient-derived glioblastoma organoid model and biobank recapitulates inter- and intra-tumoral heterogeneity. Cell 2020. 180 (1): 188-204.e22. doi: 10.1016/j.cell.2019.11.036

Kessler A F, Frombling G E, Gross F, Hahn M, Dzokou W, Ernestus R-I, Löhr M, Hagemann C. Effects of tumor treating fields (TTFields) on glioblastoma cells are augmented by mitotic checkpoint inhibition. Cell Death Discov 2018. 4:12. doi: 10.1038/s41420-018-0079-9

Kirson E D, Dbalý V, Tovarys F, Vymazal J, Soustiel J F, Itzhaki A, Mordechovich D, Steinberg-Shapira S, Gurvich Z, Schneiderman R, Wasserman Y, Salzberg M, Ryffel B, Goldsher D, Dekel E, Palti Y. Alternating electric fields arrest cell proliferation in animal tumor models and human brain tumors. Proc Natl Acad Sci i USA 2007. 104 (24): 10152-10157. doi: 10.1073/pnas.0702916104

Kirson E D, Gurvich Z. Schneiderman R, Dekel E, Itzhaki A, Wasserman Y, Schatzberger R, Palti Y. Disruption of cancer cell replication by alternating electric fields. Cancer Res 2004. 64 (9): 3288-3295. doi: 10.1158/0008-5472.can-04-0083

Kissling C. Di Santo S. Tumor Treating Fields-Behind and beyond inhibiting the cancer cell. CNS doi: Neurol Disord Drug Targets 2020. 19 (8): 599-610. 10.2174/1871527319666200702144749

Kwan K. Schneider J R, Boockvar J A. Quality of life in patients with glioblastoma treated with tumor-treating fields. JAMA 2018. 319 (17): 1822-1823. doi: 10.1001/jama.2018.1858

Lacouture M E, Davis M E, Elzinga G, Butowski N, Tran D, Villano J L, Di Meglio L, Davies A M, Wong E T. Characterization and management of dermatologic adverse events with the novoTTF-100A system, a novel anti-mitotic electric field device for the treatment of recurrent glioblastoma. Semin Oncol 2014. 41 Suppl 4: S1-14. doi: 10.1053/j.seminoncol.2014.03.011

Li L T, Jiang G, Chen Q. Zheng J N. Ki67 is a promising molecular target in the diagnosis of cancer (Review). Mol Med Rep 2015. 11:1566-1572. doi: 10.3892/mmr.2014.2914

Louis D N, Perry A, Wesseling P, Brat D J, Cree I A, Figarella-Branger D, Hawkins C. Ng H K, Pfister S M, Reifenberger G, Soffietti R, von Deimling A, Ellison D W. The 2021 WHO classification of tumors of the central nervous system: a summary. Neuro-Oncol 2021. 23 (8): 1231-1251. doi: 10.1093/neuonc/noab106

Merz F. Gaunitz F, Dehghani F, Renner C, Meixensberger J, Gutenberg A, Giese A, Schopow K, Hellwig C. Schäfer M, Bauer M, Stöcker H, Taucher-Scholz G, Durante M, Bechmann I. Organotypic slice cultures of human glioblastoma reveal different susceptibilities to treatments. Neuro Oncol 2013. 15 (6): 670-681. doi: 10.1093/neuonc/not003

Patil C G, Eboli P, Hu J. Management of multifocal and multicentric gliomas. Neurosurg Clin N Am 2012. 23 (2): 343-350. doi: 10.1016/j.nec.2012.01.012

Riley M M, San P, Lok E, Swanson K D, Wong E T. The clinical application of tumor treating fields therapy in glioblastoma. J Vis Exp 2019. 146: e58937. doi: 10.3791/58937

Rivera A L, Pelloski C E, Gilbert M R, Colman H, De La Cruz C. Sulman E P, Bekele B N, Aldape K D. MGMT promoter methylation is predictive of response to radiotherapy and prognostic in the absence of adjuvant alkylating chemotherapy for glioblastoma. Neuro Oncol 2010. 12 (2): 116-121. doi: 10.1093/neuonc/nop020. Epub 2009 Dec. 14

Rominiyi O, Vanderlinden A, Clenton S J, Bridgewater C, Al-Tamimi Y, Collis S J. Tumour treating fields therapy for glioblastoma: current advances and future direction. Br J Cancer 2020. 124 (4): 697-709. doi: 10.1038/s41416-020-01136-5

Schindelin J, Arganda-Carreras I, Frise E, Kaynig V, Longair M, Pietzsch T, Preibisch S, Rueden C, Saalfeld S, Schmid B, Tinevez J-Y, White D J, Hartenstein V, Eliceiri K, Tomancak P, Cardona A. Fiji: An open-source platform for biological-image analysis. Nat Methods 2012. 9 (7): 676-682. doi: 10.1038/nmeth.2019

Schneider C A, Rasband W S, Eliceiri K W. NIH Image to ImageJ: 25 years of image analysis. Nat Methods 2012. 9 (7): 671-675. doi: 10.1038/nmeth.2089

Schulz E, Hohmann T, Hohmann U, Ernestus R-I, Löhr M, Dehghani F, Hagemann C. Preparation and culture of organotypic hippocampal slices for the analysis of brain metastasis and primary brain tumor growth. Methods Mol Biol 2021. 2294:59-77. doi: 10.1007/978-1-0716-1350-4_5

Stupp R, Hegi M E, Mason W P, van den Bent M J, Taphoorn M J B, Janzer R C, Ludwin S K, Allgeier A, Fisher B, Belanger K, Hau P, Brandes A A, Gijtenbeek J, Marosi C, Vecht C J, Mokhtari K, Wesseling P, Villa S, Eiosenhauer E, Gorlia T, Weller M, Lacombe D, Cairncross J G, Mirimanoff R-O, European Organisation for Research and Treatment of Cancer Brain Tumour and Radiation Oncology Groups, National Cancer Institute of Canada Clinical Trials Group. Effects of radiotherapy with concomitant and adjuvant temozolomide versus radiotherapy alone on survival in glioblastoma in a randomized phase III study: 5-year analysis of the EORTC-NCIC trial. Lancet Oncol 2009. 10 (5): 459-466. doi: 10.1016/S1470-2045 (09) 70025-7

Stupp R, Mason W P, van den Bent M J, Weller M, Fisher B, Taphoorn M J B, Belanger K, Brandes A A, Marosi C, Bogdahn U, Curschmann J, Janzer R C, Ludwin S K, Gorlia T, Allgeier A, Lacombe D, Cairncross J G, Eisenhauer E, Mirimanoff R O, European Organization for Research and Treatment of Cancer Brain Tumor and Radiotherapy Groups, National Cancer Institute of Canada Trials Group. Radiotherapy plus concomitant and adjuvant temozolomide for glioblastoma. N Engl J Med 2005. 352 (10): 987-996. doi: 10.1056/NEJMoa043330

Stupp R, Tallibert S, Kanner A, Read W, Steinberg D, Lhermitte B, Toms S, Idbaih A, Ahluwalia M S, Fink K, Di Meco F. Lieberman F, Zhu J-J, Stragliotto, G, Tran D, Brem S, Hottinger A, Kirson E D, Lavy-Shahaf G, Weinberg U, Kim C-Y, Paek S-H, Nicholas G, Bruna J, Hirte H, Weller M, Palti Y, Hegi M E, Ram, Z. Effect of Tumor-Treating Fields plus maintenance temozolomide vs maintenance temozolomide alone on survival in patients with glioblastoma: a randomized clinical trial. Jama 2017. 318 (23): 2306-2316. doi: 10.1001/jama.2017.18718

Thomasova D, Anders H-J. Cell cycle control in the kidney. Nephrol Dial Transplant 2015. 30:1622-1630. doi: 10.1093/ndt/gfu395

Toms S A, Kim C Y, Nicholas G, Ram Z. Increased compliance with tumor treating fields therapy is prognostic for improved survival in the treatment of glioblastoma: a subgroup analysis of the E-14 phase III trial. J Neurooncol 2019. 141 (2): 467-473. doi: 10.1007/s11060-018-03057-z

Voloshin T, Munster M, Blatt R, Shteingauz A, Roberts P C, Schmelz E M, Giladi M, Schneiderman R S, Zeevi E, Porat Y, Bomzon Z, Urman N, Itzhaki A, Cahal S, Kirson E D, Weinberg U, Palti Y. Alternating electric fields (TTFields) in combination with paclitaxel are therapeutically effective against ovarian cancer cells in vitro and in vivo. Int J Cancer 2016. 139:2850-2858. doi: 10.1002/ijc.30406

Wick W, et al. Gliome, S2k-Leitlinie, 2021. In: Deutsche Gesellschaft für Neurologie (Hrsg.), Leitlinien für Diagnostik und Therapie in der Neurologie. Online: www.dgn/leitlinien. (accessed 16.02.2022).

We claim:

1. A method of determining the efficacy of an alternating electric field comprising:
applying an alternating electric field to a microtumor for a period of time, the alternating electric field having a frequency and field strength, wherein the microtumor comprises primary cancer cells; and
determining the efficacy of the alternating electric field.

2. The method of claim 1, wherein the microtumor is formed by seeding primary cancer cells on an organotypic tissue slice culture.

3. The method of claim 2, wherein the organotypic tissue slice culture is an organotypic hippocampal slice culture (OHSC).

4. The method of claim 2, wherein the primary cancer cells are patient derived primary cells (PDPC).

5. The method of claim 4, wherein the PDPCs are GBM-patient derived primary cells.

6. The method of claim 3, wherein the OHSC is murine OHSC.

7. The method of claim 2, wherein the organotypic tissue slice culture is cultured alone for 1-2 weeks prior to seeding the organotypic tissue slice culture with the primary cancer cells.

8. The method of claim 2, wherein seeding primary cancer cells an organotypic tissue slice culture comprises seeding between 1×10³-1×10⁴ primary cancer cells onto the surface of the organotypic tissue slice culture.

9. The method of claim 1, wherein the microtumor is formed on an organotypic tissue slice culture on a semipermeable membrane.

10. The method of claim 1, wherein determining the efficacy of the alternating electric field comprises detecting the expression of a marker associated with cell proliferation in the microtumor before applying the alternating electric field and detecting the expression of the marker in the microtumor after applying the alternating electric field, wherein a decrease in expression of the marker in the microtumor indicates the alternating electric field is effective.

11. The method of claim 1, wherein determining the efficacy of the alternating electric field comprises measuring a size of the microtumor prior to and after applying the alternating electric field, wherein a decrease in the size of the microtumor after applying the alternating electric field indicates the alternating electric field is effective.

12. The method of claim 2, further comprising applying alternating electric fields to a target site of a subject for a period of time, the alternating electric fields having a frequency and field strength,
wherein the primary cancer cells are derived from the subject,
wherein the target site comprises cancer cells.

13. A method of determining the efficacy of an alternating electric field comprising:
applying alternating electric fields to an organoid for a period of time, the alternating electric fields having a frequency and field strength; and
determining the efficacy of alternating electric fields.

14. The method of claim 13, wherein the organoid is cultured on an organotypic tissue slice culture before applying alternating electric fields.

15. The method of claim 14, wherein the organotypic tissue slice culture is an OHSC.

16. The method of claim 13, wherein the organoid is patient derived and generated from tumor tissue from the patient.

17. The method of claim 13, wherein the organoid is between 500 and 600 μm.

18. The method of claim 13, wherein determining the efficacy of the alternating electric field comprises determining the expression of a marker associated with cell proliferation, wherein a decrease in expression of the marker in the organoid indicates the alternating electric field is effective or measuring a size of the organoid prior to and after applying the alternating electrical field, wherein a decrease in the size of the organoid after applying the alternating electrical field indicates the alternating electric field is effective.

19. A method of determining the efficacy of alternating electric fields on a subject comprising
culturing a tumor slice from the subject,
applying alternating electric fields to the tumor slice for a period of time, the alternating electric fields having a frequency and field strength, and
determining the efficacy of alternating electric fields.

20. The method of claim 1, wherein the frequency of the alternating electric field is between 50 kHz and 1 MHz.
